(19) United States
(12) Patent Application Publication
(10) Pub. No.: US 2026/0259229 A1
(43) Pub. Date: Sep. 3, 2026

(54) FATIGUE SYNDROME SIGNATURES AND METHODS OF TREATMENT

(71) Applicant: The Board of Trustees of the Leland Stanford Junior University, Stanford, CA (US)

(72) Inventors: Mark M. Davis, Atherton, CA (US); Sadasivan Shankar, Menlo Park, CA (US); Vishnu Shankar, Cupertino, CA (US); Hector Bonilla, Redwood City, CA (US); Paul Mischel, Redwood City, CA (US); Michael P. Snyder, Stanford, CA (US)

(21) Appl. No.: 19/489,209

(22) PCT Filed: Jun. 5, 2024
(86) PCT No.: PCT/US2024/032556

§ 371 Date: Dec. 1, 2025

Related U.S. Application Data
(60) Provisional application No. 63/471,165, filed on Jun. 5, 2023.
(60) Provisional application No. 63/550,518, filed on Feb. 6, 2024.

(51) Int. Cl. G01N 33/68; G01N 33/564

(52) U.S. Cl. CPC G01N 33/6893; G01N 33/564; G01N 33/6863; G01N 2800/10; G01N 2800/24

(57) ABSTRACT

A common molecular signature of elevated oxidative stress is detected in blood samples from individuals with fatigue syndromes, including LC, ME-CFS, and lupus patients reporting fatigue. Analysis can be performed with one or more of flow cytometry, bulk RNA-seq analysis, mass spectrometry, and systems chemistry analysis. Elevated reactive oxygen species (ROS) were found, as well as differences in glutathione ROS clearance pathways and markers of oxidative damage.

### CROSS REFERENCE TO OTHER APPLICATIONS

This application is a U.S. National Stage and claims the benefit of PCT Application No. PCT/US2024/032556, filed, Jun. 5, 2024, which claims the benefit of U.S. Provisional Application No. 63/550,518, filed, Feb. 6, 2024 and U.S. Provisional Application No. 63/471,165, filed Jun. 5, 2023, the contents of which applications are hereby incorporated by reference in their entirety.

### STATEMENT REGARDING FEDERALLY SPONSORED RESEARCH OR DEVELOPMENT

This invention was made with Government support under contracts AI057229 awarded by the National Institutes of Health. The Government has certain rights in the invention.

### BACKGROUND

With more than 660 million documented COVID cases worldwide, it has been estimated that as many as 65 million individuals may have “Long COVID”, a complex multisystemic condition associated with post-acute sequelae of Severe Acute Respiratory Syndrome Coronavirus 2 (SARS-CoV-2) infection (PASC). Long COVID spans all ages, affecting even those who have experienced only moderate COVID infections, with 10-12% vaccinated, 10-30% of non-hospitalized, 50-70% of hospitalized COVID-19 infection survivors estimated to endure persistent symptoms after infection, with symptoms affecting individual quality of life and functional status. Although the clinical presentation of Long COVID (LC) is highly heterogeneous, with adverse events spanning multiple organ systems from dysrhythmia and higher incidence of cardiac disorders to neurological and cognitive deficits such as memory loss and “brain fog”, there are several common shared symptoms of LC, including fatigue (estimated pooled prevalence of 47%), shortness of breath (32%), and muscle pain (25%).

Strikingly, the clinical presentation LC strongly resembles myalgic encephalomyelitis/chronic fatigue syndrome (ME/CFS), a complex chronic disease estimated to affect between 836,000 to 2.5 million individuals in the US alone. By meta-analysis, it is estimated that 0.01 to 7.62% of the world population has ME/CFS, with 2× higher preponderance among females compared to males. Based on three symptoms defined by the National Academy of Medicine criteria, ME/CFS is characterized by (1) profound fatigue lasting for at least 6 months, (2) post-exertional malaise, and (3) unrefreshing sleep. Prospective studies have noted around half of the patients with LC meet the diagnostic criteria for ME/CFS, and roughly 75% of ME/CFS patients report an infection preceding symptom onset.

Despite these similarities in clinical symptoms, there is no known molecular basis of ME/CFS and LC, with few available molecular signatures that explain the shared symptoms. Due to the lack of available molecular markers, there are neither standard diagnostic tests nor treatments for LC and ME/CFS. With no available options, patients with ME/CFS and/or LC can only be diagnosed with a physical and mental examination, based on the presentation of symptoms such as persistent fatigue and post-exertional malaise. With the mounting public health burden of ME/CFS and LC, there is an urgent need to understand the biochemical underpinnings of these conditions, which can guide the development of new diagnostics and therapies.

### SUMMARY

Compositions and methods are provided for determining the presence of a fatigue syndrome in an individual by non-invasive analysis of a sample, e.g. a blood sample comprising circulating immune cells. Fatigue syndromes include, without limitation, long Covid (LC), myalgic encephalomyelitis or chronic fatigue syndrome (ME/CFS), and certain autoimmune diseases. It is shown that oxidative stress features associated with fatigue syndrome are present in circulating immune cells, which cells can be utilized for diagnosis and therapeutic stratification of individuals. The oxidative stress features thus identified also provide the basis for compound screening and therapeutic methods. In some embodiments the individual for diagnosis, screening or therapy is a human. In some embodiments the individual for diagnosis, screening or therapy is a human female.

It is shown herein that a common molecular signature of elevated oxidative stress can be detected in samples of blood cells from individuals with fatigue syndromes, including LC, ME-CFS, and lupus patients reporting fatigue. Detection of these markers can be performed with one or more of flow cytometry, bulk RNA-seq analysis, mass spectrometry, and systems chemistry analysis. Elevated reactive oxygen species (ROS) in lymphocytes were found to be associated with fatigue syndromes, as well as differences in glutathione ROS clearance pathways, and markers of oxidative damage, including lipid peroxidation. Hyperproliferation of lymphocytes was also associated with these conditions. These findings highlight mitochondrial dysfunction, lipid peroxidation, and lymphocyte hyperproliferation as markers for patient identification and stratification, which provide targetable pathways for ME-CFS and LC drug screening and therapeutic intervention.

In some embodiments, following immune cell profiling for oxidative stress and a determination that a fatigue syndrome is present, the individual is treated to ameliorate, diminish, or actively treat the syndrome. Such treatment may prevent progression, or reduce severity, of the fatigue syndrome. In some embodiments treatment is pharmacologic, for example administering therapeutic agents that modify ROS pathways. In some embodiments a therapeutic agent is identified by a screening method disclosed herein. In other embodiments treatment comprises diet, physical and/or occupational therapy. In some embodiments treatment comprises clinical trial enrollment, where individuals can be stratified by their likelihood of a fatigue syndrome diagnosis. The oxidative stress markers can be employed as endpoints for studying drug effects in these conditions, where therapies aim to relieve patient fatigue by mitigating existing oxidative damage and/or restoring the balance between oxidative stress/anti-oxidants for improved function of the individuals.

In some embodiments, a sample comprising immune cells, e.g. a blood sample, PBL sample, etc. from an individual is contacted with a detectable agent, including without limitation a dye for the presence of Ca²⁺; dyes for tracing proliferation; dyes for detecting the presence of reactive oxygen species; dyes for detecting lipid species or lipid droplets; affinity agents specific for protein markers of interest, etc. The set of markers identified by the detectable agent(s) is sufficient to phenotype at least one, at least two, at least three, at least four, and may be five or more fatigue syndrome biomarkers, e.g. ROS, lipids, lipid droplets, T cell markers, B cell markers, gene expression, cell proliferation, Ca²⁺ levels, etc. The sample is analyzed for the presence of the detectable marker(s), and compared against a reference or healthy control sample, for a determination of the presence of a fatigue syndrome. Cell samples may be gated in analysis, e.g. by flow cytometry, mass cytometry, etc., on immune cell populations, such as CD19⁺ B cells, CD4⁺ T cells, CD8⁺ T cells, etc.

In some embodiments, a biomarker of a fatigue syndrome is determination of total reactive oxygen species levels in an immune cell sample, where there is an increase relative to a healthy control, e.g. an increase of greater than about 1.2-fold, greater than about 1.5-fold, greater than about 1.7-fold, or more. In some embodiments the presence of ROS is determined by contacting cells with a fluorogenic dye that reacts with oxygen species, including without limitation dichlorodihydrofluorescein diacetate (DCFDA). The cells are conveniently analyzed by flow cytometry for the presence of fluorescence, e.g. for median and/or maximum fluorescence intensity.

In some embodiments, a biomarker of a fatigue syndrome is determination of the balance between oxidative stress and anti-oxidant pathways in an immune cell sample. In some embodiments the biomarkers are the presence of one or more of: glutathione (GSH) levels; the ratio of mitochondrial Ca²⁺ to superoxide dismutase 2 (SOD2); and expression of catalase. In some embodiments the presence of one or more of these biomarkers is determined by contacting cells with a detectably labeled agent that reacts with glutathione, with catalase, with SOD2, with Ca++, etc. The cells are conveniently analyzed by flow cytometry for the presence of fluorescence, e.g. for median and/or maximum fluorescence intensity. Increased glutathione, increased catalase, and ratio elevation across lymphocytes for mitochondrial Ca²⁺:SOD2 relative to a healthy control is indicative of a fatigue-associated syndrome, e.g. an increase of greater than about 1.2-fold, greater than about 1.5-fold, greater than about 1.7-fold, or more. Alternatively, expression of genes of interest, such as catalase or SOD2 can be performed at the RNA level, e.g. RNA-seq analysis and the like.

In some embodiments, a biomarker of a fatigue syndrome is determination of total cellular oxidative damage in an immune cell sample, for example by the measurement of lipid-related biomarkers. In some such embodiments the presence of glutathione peroxidase 4 (GPX4) protein or GPX4 transcripts is determined by contacting cells with a detectably labeled agent that reacts with GPX4. The cells are conveniently analyzed by flow cytometry for the presence of fluorescence, e.g. for median and/or maximum fluorescence intensity where there is an increase in disease samples relative to a healthy control, e.g. an increase of greater than about 1.2-fold, greater than about 1.5-fold, greater than about 1.7-fold, or more. In other embodiments the biomarker may comprise a direct comparison of lipid peroxidation through flow cytometry, changes in lipid droplet levels as evaluated through flow cytometry and/or immunofluorescence analysis; changes in lysophosphatidylethanolamine (lysoPE) phospholipid levels, detection through mass spectrometry; and the like.

In some embodiments, a biomarker of a fatigue syndrome is increased lymphocyte proliferation in response to stimulus. In some such embodiments the lymphocyte proliferation is determine by labeling the a cell sample comprising lymphocytes with a tracer dye, e.g. CellTrace™, CSFE, CMAC, Blue CMF2HC, Violet BMQC, Green CMFDA, Orange CMRA, CM-Dil, CMTPX, Deep Red, etc. The labeled cells are exposed to a stimulus, e.g. anti-CD3, IL-2 and anti-CD28; allogeneic cells; antigen; etc. The level of proliferation can be determined by dye levels, e.g. by flow cytometry, where there is an increase in disease samples relative to a healthy control, e.g. an increase of greater than about 1.2-fold, greater than about 1.5-fold, greater than about 1.7-fold, or more in lymphocyte proliferation.

In some embodiments a panel of biomarkers analyzed in a lymphocyte population comprises at least one, two, three, four, five, or all of (a) determination of ROS by flow cytometry; (b) determination of the ratio of mitochondrial Ca++ to SOD2 mean fluorescence intensity by flow cytometry; (c) determination of the maximum glutathione (GSH) level by flow cytometry; (d) analyzing by flow cytometry the level of glutathione peroxidase 4 (GPX4); (e) determination of the presence of lipid peroxides; (f) determination of the presence of lipid droplets; and (g) determination of lymphocyte proliferation in response to stimuli.

The detectably labeled population in any of the above assays may be analyzed by flow cytometry. In some embodiments, the flow cytometry is fluorescence activated flow cytometry. In some embodiments at least 10³ cells; at least 10⁴ cells; at least 10⁵ cells are analyzed. In some embodiments the cell sample is gated on lymphocyte populations, e.g. using labeled affinity reagents. The resulting dataset may be input into a predictive classification algorithm for a determination of whether the individual has a fatigue syndrome.

The analysis may further comprise analysis other than immune cell profiling, e.g. clinical indicia including a patient response questionnaire for indications of fatigue, and compared to a control or reference value.

In one embodiment of the invention, the methods of determining the presence of a fatigue syndrome in an individual comprise obtaining a patient sample comprising circulating immune cells. Blood samples are a convenient source of circulating immune cells, particularly whole blood, although PBMC fractions also find relevant use. The sample(s) is (are) physically contacted with a panel of reagents specific for the panel of biomarkers as described above. The reagent specific for a biomarker may be, for example, affinity reagents, Ca²⁺ dyes, oxygen reactive dyes, etc. comprising a detectable label, e.g. isotope, fluorophore, etc. Signal intensity of the markers can be measured at a single cell level or in bulk. The data may be clustered and compared to measurements of the same from a training population. The data can be normalized for comparisons between different samples. An individual determined to have a fatigue syndrome may be treated to ameliorate, diminish, or actively treat the syndrome. Such treatment may prevent progression or reduce severity of the fatigue syndrome. In some embodiments treatment is pharmacologic, for example including administration of an effective dose of therapeutic agents that modify ROS pathways, such as metformin, etc. In some embodiments a therapeutic agent is identified by a screening method disclosed herein.

Also described herein is a method for determining the presence of a fatigue syndrome in an individual, comprising: obtaining a dataset associated with an immune sample obtained from the individual, wherein the dataset comprises quantitative data from the biomarkers disclosed herein and analyzing the dataset classification relative to a predictive model, wherein a statistically significant match with a model disclosed herein is indicative of fatigue syndrome. The data may be analyzed by a computer processor. The processor may be communicatively coupled to a storage memory for analyzing the data. The processor may be coupled to a flow cytometer, and may include algorithms for clustering cell populations, and predictive classification. Also described herein is a computer-readable storage medium storing computer-executable program code, the program code comprising: program code for storing and analyzing data obtained by the methods of the disclosure.

In an embodiments, methods are provided for screening candidate agents for treatment of a fatigue syndrome, for example as shown in FIG. 9. A screening assay of interest utilizes cell-based assays for determining an effect on lymphocyte proliferation, and oxidation stress parameters. Cells may be initially tested, for example, to screen candidate agents for ROS-protective, or ROS-reversing effects. In such an assay, an immune cell population is exposed to a candidate agent before or after exposure to an agent that induces ROS damage. The effect of the candidate agent may be monitored by at least one, two, three, four, five, or all of (a) determination of ROS by flow cytometry; (b) determination of the ratio of mitochondrial Ca++ to SOD2 mean fluorescence intensity by flow cytometry; (c) determination of the glutathione (GSH) level by flow cytometry; (d) analyzing by flow cytometry the level of glutathione peroxidase 4 (GPX4); (e) determination of lipid peroxides; (f) determination of lipid droplets; and (g) lymphocyte proliferation in response to stimuli. Candidate agents with ROS-protective or ROS-reversing activity may be further tested on a population of lymphocytes from an individual with a fatigue disorder in an assay as described above, to determine the effectiveness in modifying oxidative stress and proliferative parameters. Agents with activity can be used in treatment of individuals with a fatigue disorder.

In other embodiments of the invention a device or kit is provided for the analysis of patient samples. Such devices or kits will include reagents that specifically identify one or more immune cells; and detectable markers for least one, two, three, four, five, or all of (a) determination of ROS by flow cytometry; (b) determination of the ratio of mitochondrial Ca++ to SOD2 mean fluorescence intensity by flow cytometry; (c) determination of the maximum glutathione (GSH) level by flow cytometry; (d) analyzing by flow cytometry the level of glutathione peroxidase 4 (GPX4); (e) determination of lipid peroxides; (f) determination of lipid droplets; and (g) lymphocyte proliferation in response to stimuli. The reagents can be provided in isolated form, or pre-mixed as a cocktail suitable for the methods of the invention. A kit can include instructions for using the plurality of reagent s to determine data from the sample; and instructions for statistically analyzing the data. The kits may be provided in combination with a system for analysis, e.g. a system implemented on a computer. Such a system may include a software component configured for analysis of data obtained by the methods of the invention.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1D. Comparison of total reactive oxygen species (ROS) levels in lymphocytes of healthy controls (HC), myalgic encephalomyelitis-chronic fatigue syndrome (ME/CFS), and Long COVID (LC) donors. (A) Peripheral blood mononuclear cells were characterized from 16 healthy control (HC), 15 Long COVID (LC), and 15 Myalgic Encephalomyelitis/Chronic Fatigue Syndrome (ME/CFS) samples. Age distributions were similar between all groups with gender distributions similar between HC and LC groups. (B) Representative flow cytometry plots for reactive oxygen species (ROS) indicator 2′,7′-dichlorodihydrofluorescein diacetate (DCFDA) are shown for CD4 T cells for a single healthy control (HC), Long COVID (LC), and ME/CFS donor. Y-axis represents normalized events to the mode. For C-E, * indicates p<0.05, ** indicates p<0.01, *** indicates p<0.001 based on a two-sided t-test (C) Comparing 16 HC, 15 LC, and 15 ME/CFS donor lymphocytes, DCFDA staining shows higher total median fluorescence intensity (MFI) ROS levels in ME/CFS (CD4T p=5.60*10-4, CD8T p=1.91*10-4, CD19B p=3.04*10-4) and LC (CD4 T cells p=0.104, CD8 T cells p=0.0210, CD19 B cells p=0.0727) lymphocytes compared to healthy controls. (D) Comparison of MFI ROS levels uniquely identifies higher ROS levels in ME/CFS and LC females, compared to controls (females ME/CFS v. HC: CD19 B cells p=0.00065, CD4 T cells p=0.00045, CD8 T cells p=9.5*10-5; LC v. HC: CD19 B cells p= 0.144, CD4 T cells p=0.062, CD8 T cells p=0.01065). In contrast, ME/CFS and LC males show no significant difference (males ME/CFS v. HC: CD19 B cells p=0.357, CD4 T cells p=0.469, CD8 T cells p=0.516; males ME/CFS v. LC: CD19 B cells p=0.278, CD4 T cells p=0.643, CD8 T cells p=0.583).

FIGS. 2A-2E. Comparison of important intermediates and proteins involved in ROS generation and clearance within lymphocytes between HC, LC, and ME/CFS donors. (A) Summary of ROS generation and clearance pathways, including mitochondrial ROS (e.g., superoxide O2−) calcium and NADPH oxidase-mediated generation, superoxide dismutase mediated-conversion to hydrogen peroxide, and hydrogen peroxide breakdown by catalase and glutathione. The alterations in these ROS generation and clearance pathways among ME/CFS, LC donors are shown by sex (females shown in brown, males in gray). Upward arrow corresponds to elevation of parameter in ME/CFS and LC donors, compared to healthy controls of the same gender. The rest of FIG. 2 and FIG. 3 includes the data supporting these sex-specific differences. For B-D, * indicates p<0.05, ** indicates p<0.01, *** indicates p<0.001 based on a two-sided t-test (B) Comparison of mitochondrial calcium levels based on Rhod-2 AM flow cytometric staining identifies statistically significant higher levels in ME/CFS and LC female donors (females: ME/CFS v. HC CD19B p=0.00238, CD4T p=0.0063, CD8T p=0.0137; ME/CFS v. LC CD19B p=0.0097, CD4T p=0.0172, CD8T p=0.240; males: ME/CFS v. HC CD19B p=0.791, CD4T p=0.514, CD8T p=0.825; LC v. HC CD19B p=0.851, CD4T p=0.241, CD8T p=0.358). (C) Flow cytometric comparison of mitochondrial superoxide dismutase 2 (SOD2) protein levels finds significantly lower SOD2 levels in LC donors, compared to healthy controls (HC v. ME/CFS CD19 B cells p=0.326, CD4 T cells p=0.638, CD8 T cells p=0.384; HC v. LC CD19 B cells p=0.073, CD4 T cells p=0.023, CD8 T cells p=0.023). (D) Comparison of glutathione (GSH) levels finds significantly higher MFI levels in both male and female ME/CFS donors (females ME/CFS v. HC CD19 B cells p=0.00048, CD4 T cells p=0.00014, CD8 T cells p=0.00159; LC v. HC CD19B cells p=0.580, CD4 T cells p=0.827, CD8T cells p=0.958; males ME/CFS v. HC CD19 B cells p=0.0327, CD4 T cells p=0.0354, CD8 T cells p=0.0894; LC v. HC CD19 B cells p=0.196, CD4 T cells p=0.093, CD8 T cells p=0.278). (E) Based on the reaction between hydrogen peroxide (H2O2) and glutathione (GSH) in FIG. 2A (H2O2+2GSH←2H2O+GSSG), the association between GSH and total ROS levels (FIG. 1C) is shown for both females (top row) and males (bottom row). With each point corresponding to a donor, the plot shows a statistically significant positive correlation between total ROS and GSH levels (females CD19 B cells R=0.56, p=0.0039, CD4 T cells R=0.63 p=6.9*10-4; CD8 T cells R=0.52 p=7.8*10-3; males CD19 B cells R=0.5 p=0.025, CD4 T cells R=0.56 p=0.01, CD8 T cells R=0.43 p=0.057).

FIGS. 3A-3H. Identification of lipid oxidative damage in ME/CFS and LC donor lymphocytes, compared to healthy controls. (A) Using extracted metabolites from 200,000 sorted CD3 T cells from ME/CFS and HC donors, hydrophilic interaction liquid chromatography-mass spectrometry and Turium analysis were conducted to map metabolic pathway differences between ME/CFS donor and healthy control T cells. From 747 identified metabolites, the top five mapped pathway differences are shown. Size of the nodes correspond to magnitude of the difference in relative abundance of detected metabolites from mass spectrometry between ME/CFS and HC samples. (B) The reactions encoded by the large red circles (FIG. 3A) are shown, corresponding to reactions significantly up-regulated in CFS donors compared to healthy controls. The phospholipid synthesis reactions involve the production of lysoPE metabolites and are catalyzed by phospholipase A2 (PLA2). (C) Comparison of PLA2 transcript levels from bulk RNA-seq data (Bouquet et al., Ref. 42) between 11 sedentary controls and 14 CFS patients identifies significantly higher levels of PLA2 in CFS donors (p<0.05, two-sided t-test). Transcript levels are compared using fragments per kilobase of transcript per million mapped reads (FPKM). (D) Representative images from immunofluorescence staining are shown for glutathione peroxidase 4 (GPX4) in CD3 T cells from 1 HC, 1 LC, and 1 ME/CFS donor (blue corresponds to DAPI, red to CD3, magenta to MitoTracker Far Red/mitochondria, green to GPX4). (E) Quantification of average GPX4 pixel intensity from 1 HC, 1 LC, and 1 ME/CFS donor. Each point corresponds to a CD3 T cell, with comparisons conducted across 426 HC, 603 ME/CFS, and 239 LC T cells from maximum projection images. Significant increases in average pixel intensity indicate higher GPX4 levels in ME/CFS and LC donors, compared to healthy controls (p<2.2*10⁻¹⁶ for both ME/CFS and LC from two-sided t-test). (F) Flow cytometric comparison of lipid peroxide levels, using ratiometric lipid peroxidation sensor, shows both ME/CFS and LC donor lymphocytes have significantly higher lipid peroxidation, compared to healthy controls. Due to lower lipid peroxide levels in male healthy controls, male ME/CFS and LC donors exhibit larger changes compared to females (males ME/CFS v. HC: CD19 B cells p=0.0027, CD4 T cells p=0.0037, CD8 T cells p=0.0041, LC v. HC CD19 B cells p=0.003, CD4 T cells p=0.0033, CD8 T cells p=0.0029; females ME/CFS v. HC: CD19 B cells p=0.076, CD4 T cells p=0.448, CD8 T cells p=0.131, LC v. HC: CD19 B cells p=0.0365, CD4 T cells p=0.236, CD8 T cells p=0.0977). Lower y-axis values correspond to higher lipid peroxidation. (G) Flow cytometric comparison of lipid droplet levels, using HCS LipidTox Green Neutral Lipid droplet staining, identifies lower lipid droplet levels in ME/CFS donors compared to HC across both males and females (males ME/CFS v. HC: CD19 B cells p=1.52*10⁻⁴, CD4 T cells p=5.92*10⁻⁴, CD8 T cells p=7.14*10⁻⁴, LC v. HC: CD19 B cells p=7.31*10⁻³, CD4 T cells p=9.39*10⁻³, CD8 T cells p=0.0114; females ME/CFS v. HC CD19 B cells p=0.0242, CD4 T cells p=0.0205, CD8 T cells p=0.00876; LC v. HC CD19 B cells p=0.622, CD4 T cells p=0.826, CD8 T cells p=0.974). (H) Negative association between lipid droplet (FIG. 3G) and GSH levels (FIG. 2D) among all lymphocyte subsets (females: CD19 B cells R=−0.63, p=0.0072, CD4 T cells R=−0.67 p=0.003, CD8 T cells R=−0.54 p=0.025; males: CD19 B cells R=−0.9, p=2.5*10⁻⁵, CD4 T cells R=−0.75 p=0.0032, CD8 T cells R=−0.78, p=0.0015).

FIGS. 4A-4F. Effects of ROS on Adaptive Immune Response. (A) Representative glutathione (GSH) profile from flow cytometry staining for a HC, LC, and ME/CFS CD4 T cell shows a long-tailed distribution for both HC and LC donors and a shifted distribution for ME/CFS. (B) To study tail phenomena, the distribution of 95th percentile from flow cytometry GSH data is plotted across HC, LC, and ME/CFS donors. Each group demonstrates distinct tail-decaying behavior, where healthy controls decay quickly (light-tailed distribution), LC show heavy-tailed decay, and ME/CFS show a shifted and bounded tail distribution. (C) To understand this decaying behavior at the tail of the distribution, we compared the GSH levels on an additional set of 5 HC, 4 LC, and 5 ME/CFS donors between total, naïve (CCR7+CD45RO−), and memory CD4 T cell populations (CD45RO+). The long tails are found only in memory and not naïve CD4 T cells, indicating that memory T cells are the main contributors to high oxidative stress in T cells. For D-F, to probe functional effects of oxidative stress, PBMCs were labeled with CellTraceViolet and stimulated with anti-CD3/CD28 beads and IL-2. Based on a dye dilution assay, the proportion of proliferating T cells as a function of total ROS levels are shown at 5 days post-stimulation. Experiments for D-F were conducted separately. (D) Data is shown for a set of 5 control and 5 ME/CFS females, for which female ME/CFS T cells hyperproliferate and exhibit higher ROS levels compared to control counterparts. The regression lines also show that control female CD4 and CD8 T cell proliferation has 10× greater slope with respect to ROS, compared to ME/CFS females. Compared to controls, the flat slope and higher line for ME/CFS T cells highlights hyperproliferation, suggesting a potential source of patient fatigue. (E) Consistent with FIG. 4D, data shown for 4 control and 5 LC females shows hyperproliferation among LC CD4 and CD8 T cells. The regression lines also show that control female CD4 and CD8 T cell proliferation has 10× greater slope with respect to ROS, compared to LC females. Compared to controls, the flat slope and higher line for LC T cells highlights hyperproliferation, consistent with the data in FIG. 4D. (F) Data shown for 7 control and 5 ME/CFS males shows a distinct pattern from females. CD4 and CD8 T cells from ME/CFS males do not hyperproliferate compared to controls, as ME/CFS males with similar ROS levels to controls do not show higher proliferation. As T cell proliferation among controls show 3.5× and 2.3× greater slope compared to ME/CFS CD4 and CD8 T cell counterparts, these results show greater insensitivity to ROS levels among ME/CFS male donors.

FIGS. 5A-5F. Identification of CFS patients using ROS signatures and tracking T cell proliferation enables personalized selection of ROS-lowering drugs to redress T cell hyperproliferation in CFS donors. (A) Study of fPOP patients flagged patients 27 and 31, along with the father of 27, based on measured total ROS levels (DCFDA) in lymphocytes. Patient clinical information shown in table identifies 27 and 31 as ME/CFS patients, based on the National Academy of Medicine ME/CFS diagnostic criteria. (B) Using CellTraceViolet staining, the proportion of proliferating T cells 5-days after stimulation were assessed for fPOP donors. CFS donor CD4 and CD8 T cells with higher ROS are also associated with higher proportion of proliferation, consistent with FIG. 4E. Proportion of proliferating T cells were determined, based on same methods in FIG. 4D. (C) To reduce proportion of proliferating T cells, PBMCs were treated at day 0 with ROS-modulating drugs, including N-acetylcysteine, metformin, and liproxstatin-1. Effects of drug on proliferation and ROS levels are shown 5-days after stimulation, where drugs reduce the proportion of high ROS proliferating T cells in Q1. (D) Comparison of drug effects between donor 27 and donor 27's mother and father shows tested ROS-lowering drugs selectively modulate T cell proliferation in the CFS donor 27 and not in the parents. (E) Extension of metformin treatment to 5 additional HC and 5 ME/CFS donors finds a statistically significant reduction in the proliferation of ME/CFS CD4 T cells (p=0.041). The drug does not alter proliferation in HC donors, highlighting its selectivity in ME/CFS donors. (F) Summary of Precision Medicine methodology for identifying ME/CFS patients and ROS-modulating drugs to address oxidative stress and hyperproliferation of T cells.

FIG. 6. Summary of proportion of autoimmune patients that experience fatigue. Example 2 in the “Experimental Section” demonstrates application of the disclosed approach for helping diagnose and identify potential treatments for systemic lupus erythematosus (SLE).

FIG. 7. Comparison of total ROS levels based on median fluorescence intensity (MFI) from DCFDA flow cytometric analysis between 5 HC, 5 ME-CFS, 5 LC, and 3 SLE donors. * denotes p<0.05, ** denotes p<0.01, *** denotes p<0.001 in a two-sided t-test. Comparison of total ROS levels using DCFDA staining shows SLE patients have higher ROS levels compared to healthy controls but lower levels compared to ME-CFS and LC donors.

FIG. 8. Comparison of lipid peroxidation median fluorescence intensity (MFI) ratio from flow cytometric analysis between 5 HC, 5 ME-CFS, 5 LC, and 3 SLE donors. A lower MFI ratio corresponds to higher lipid peroxidation or oxidative damage. * denotes p<0.05 in a two-sided t-test. Similar to total ROS levels, flow cytometric evidence suggests higher lipid peroxidation in SLE donors compared to healthy controls but lower compared to ME-CFS and LC donors.

FIGS. 9A-9F. Screening assays. (A) To identify drugs that can protect immune cell populations from oxidative stress, PBMCs are treated with candidate drugs (e.g., liproxstatin-1, metformin) for an hour, before treatment with ROS inducer (e.g., cumene hydrogen peroxide, tert-butyl hydroperoxide). By subsequently labeling cells with metabolic dyes to track oxidative stress parameters, comparisons with immune cells only treated with ROS inducer enable quantification of drugs capacity to protect against oxidative stress and damage in immune cell populations. (B) To identify drugs that can reverse oxidative stress and damage in immune cell populations, PBMCs are treated first with a ROS inducer for 30 minutes to an hour, before subsequent treatment with candidate drugs. By labeling with metabolic dyes and comparing the drug treated immune cell populations to those treated only with ROS inducer, we can quantify the capacity for drugs to reverse oxidative stress and damage in PBMCs. (C) To link these metabolic changes with immune cell function, control and disease PBMCs (e.g., from fatigue-syndrome patients) are labeled with proliferation dyes, upon which T cells are stimulated with anti-CD3/anti-CD28 antibodies and IL-2. By treating PBMCs with drugs identified from methodologies described in 9A and 9B, we can assess how metabolic changes to oxidative stress levels in drugs also modulate functional capacity of donor T cells to proliferate. While flowchart is specific to T cells (e.g., anti-CD3/anti-CD28 stimulation), the core methodology is applicable to other immune cell populations, where changes in immune cells capacity to proliferate can be assessed upon treatment with candidate drugs. (D) Application of methodology to a specific CFS patient is shown, where PBMCs treated with ROS-modulating drugs including N-acetylcysteine, metformin, and liproxstatin-1. Flow cytometry data is shown for CD4 T cells, where the x-axis corresponds to CellTraceViolet (proliferation dye) staining and the y-axis corresponds to total ROS levels. Based on higher ROS levels in CFS donors, this data shows that treatment with ROS modulating drugs also modulates hyperproliferation. (E) Proportion of proliferating T cells are shown for a specific CFS patient (patient 27) along with their mother and father. The figure shows that ROS modulating drugs appear to have a specific effect in lowering the proportion of proliferating T cells in the CFS donor (red) and not the parental healthy controls (blue). (F) This figure demonstrates the methodology in 9B, where several ROS-modulating candidate drugs are identified based on capacity for reversing oxidative damage in contrast to the positive control immune cell populations (shown in blue). Lipid peroxidation changes are shown, where higher oxidative damage corresponds to a lower value on the y-axis. Identified drugs include N-acetylcysteine (NAC), metformin, liproxstatin-1, nicotinamide mononucleotide (NMN), oxaloacetate, MitoQ, Abilify, Colchicine, Naltrexone. Although this methodology is shown for CD4 T cells, it is broadly applicable to other immune cell populations (e.g., myeloid cells).

FIGS. 10A-B. (A) Sample gating strategy is shown for CD19 B cells, CD4 and CD8 T cells. (B) After gating for CD4 T cells, CCR7 and CD45RO surface markers are used to define memory and naïve populations.

FIGS. 11A-C. Comparison of mitochondrial parameters between healthy controls (HC), ME-CFS, and LC donors. While ME-CFS and LC donors appear to present with lower mitochondrial ATP across lymphocyte populations compared to HC donors (A), no differences in mitochondrial mass (B) or membrane potential (C) are observed.

FIGS. 12A-D. Comparison of total ROS levels based on patient characteristics. (A) No difference in Total ROS levels (MFI) by fatigue severity (B) Association between Total ROS levels (MFI) and duration of LC symptoms. (C) No association with age. (D) Association with BMI in Long COVID group, where donors with higher BMI have higher total ROS levels.

FIGS. 13A-D. Comparison of redox parameters between HC, ME-CFS, and LC donors across males and females. (A) Comparison of mitochondrial calcium levels identifies statistically significant differences uniquely in females and not in males (two-sided t-test: females: CD4T ME-CFS p=0.0063, CD4T LC p=0.0172, CD8 T CFS p=0.0137, CD8T LC p=0.24, CD19 B CFS p=0.0024, CD19 B LC p=0.0097). (B) LC donors across both males and females show decreases in SOD2 levels, compared to HCs of the same gender. (C) Comparison of lipid peroxide levels are shown, where lower y-axis values correspond to higher levels of lipid peroxides. ME-CFS and LC donors across genders exhibit similar levels of lipid peroxides. As HC males have lower levels of lipid peroxides compared to HC females, the differences in lipid peroxidation are more pronounced. (D) Comparison of lipid droplet levels are shown between groups, where ME-CFS and LC donors show decreases in lipid droplets compared to HCs across both genders. Similar to lipid peroxide comparisons, HC males have higher levels of lipid droplets compared to HC females, meaning differences in ME-CFS and LC groups are greater for males.

FIGS. 14A-14F. Comparison of CD69, CD137 MFI levels between HC and ME/CFS CD4 and CD8 T cells. These differences are shown 5-days post-activation with anti-CD3/anti-CD28 antibodies and IL-2. (A, B) Plots are shown for female HC and ME/CFS donors. (C, D) Plots are shown for male HC and ME/CFS donors (E, F) Plots are shown for female HC and LC donors.

FIGS. 15A-B. Treatment of HC and ME-CFS PBMCs with (A) 100 μM N-acetylcysteine and (B) 1 M liproxstatin-1. The proportion of proliferating T cells is shown 5-days post activation. While no difference or decrease in proliferating T cells are observed upon NAC treatment (A), liproxstatin-1 treatment, which targets GPX4, selectively lowers proliferation in ME-CFS and not the HC donors.

FIGS. 16A-16C. Treatment of HC and female LC PBMCs with (A) 100 μM N-acetylcysteine (B) 10 μM metformin (C) 1 μM liproxstatin-1.

FIGS. 17A-C. Treatment of HC and male ME/CFS PBMCs with (A) 100 μM N-acetylcysteine (B) 10 μM metformin (C) 1 μM liproxstatin-1.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

Fatigue Syndrome. As used herein, the term “fatigue syndrome” refers to a chronic illness characterized by extreme fatigue. Such syndromes are difficult to diagnose, and can therefore benefit from the methods disclosed herein. Generally an individual will have “core” symptoms, including (a) greatly lowered ability to do activities that were usual before the illness. This drop in activity level occurs along with fatigue and must last six months or longer. (b) Worsening of ME/CFS symptoms after physical or mental activity that would not have caused a problem before illness, known as post-exertional malaise (PEM). (c) Sleep problems. Conditions that are often classified as fatigue syndromes include long Covid (LC); chronic fatigue syndrome; and certain autoimmune diseases, including without limitation SLE.

Some people who have been infected with the virus that causes COVID-19 can experience long-term effects from their infection, known as Long COVID or Post-COVID Conditions (PCC). Long COVID is broadly defined as signs, symptoms, and conditions that continue or develop after initial COVID-19 infection. Long COVID is a wide range of new, returning, or ongoing health problems that people experience after being infected with the virus that causes COVID-19. These health problems persist for many months after individuals test negative for SARS-CoV-2 with a polymerase chain reaction (PCR) or antigen test.

A provisional definition of LC may be the presence of persistent symptoms and sequelae beyond four weeks from onset of infection, of which the main features are breathlessness, cognitive impairment, fatigue, anxiety and depression. The often mentioned “brain fog” is characterised by difficulties with concentration, memory and executive function. Post-viral syndrome is more common in depressed patients but can occur after a number of viral infections, for example EBV, HSV and HTLV (Burrell et al., 2017). Reports of the prevalence of ongoing symptoms after COVID infection range from 32.6% to 87% of hospitalised patients. In a non-hospitalised cohort, 37% report fatigue and 30% cognitive impairment. The cause of this post-viral syndrome is not known, though it does resemble chronic fatigue syndrome, now called post viral fatigue syndrome (PVFS).

Myalgic encephalomyelitis/chronic fatigue syndrome (ME/CFS) is a disabling and complex illness. People with ME/CFS have overwhelming fatigue that is not improved by rest and suffer from post-exertional fatigue. Other symptoms can include problems with sleep, thinking and concentrating, pain, and dizziness. People with ME/CFS may not feel better or less tired, even after a full night of sleep. Some people with ME/CFS may have problems falling asleep or staying asleep. At least one in four ME/CFS patients is bed- or house-bound for long periods during their illness. An estimated 836,000 to 2.5 million Americans suffer from ME/CFS, but about 90 percent of people with ME/CFS have not been diagnosed, given the absence of specific markers or tests.

Systemic lupus erythematosus is a chronic, multisystem, inflammatory disorder of autoimmune etiology, occurring predominantly in young women. Common manifestations may include arthralgias and arthritis, Raynaud syndrome, malar and other rashes, pleuritis or pericarditis, renal or central nervous system involvement, and autoimmune cytopenias. Diagnosis requires clinical and serologic criteria. Clinical findings vary greatly. SLE may develop abruptly with fever or insidiously over months or years with episodes of arthralgias and malaise. Vascular headaches, epilepsy, or psychoses may be initial findings. Manifestations referable to any organ system may appear. Periodic exacerbations (flares) may occur. Neurologic symptoms can result from involvement of any part of the central or peripheral nervous system or meninges. Mild cognitive impairment is common. There may also be headaches, personality changes, ischemic stroke, subarachnoid hemorrhage, seizures, psychoses, aseptic meningitis, peripheral and cranial neuropathies, transverse myelitis, choreoathetosis, or cerebellar dysfunction.

The etiology of fatigue in the SLE population is multifactorial and is associated with physical activity, obesity, sleep quality, depression, anxiety, mood, cognitive dysfunction, vitamin D deficiency/insufficiency, comorbidities such as fibromyalgia, or related to the SLE disease itself or the treatments used to manage SLE. Sleep disturbances in patients with SLE are common and prevalence can be as high as 91.5%. Patients with SLE report poor sleep quality including frequent awakenings and restlessness.

The terms “subject,” “individual,” and “patient” are used interchangeably herein to refer to a mammal being assessed for treatment and/or being treated. In an embodiment, the mammal is a human. Subjects may be human, but also include other mammals, particularly those mammals useful as laboratory models for human disease, e.g. mouse, rat, etc.

The definition of an appropriate patient sample encompasses blood and other liquid samples of biological origin, solid tissue samples such as a biopsy specimen or tissue cultures or cells derived there from and the progeny thereof. The definition also includes samples that have been manipulated in any way after their procurement, such as by treatment with reagents; washed; or enrichment for certain cell populations, such as lymphocytes, etc. A sample of interest is peripheral blood. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's sample cell, e.g., a sample comprising polynucleotides and/or polypeptides that is obtained from a patient's sample cell (e.g., a cell lysate or other cell extract comprising polynucleotides and/or polypeptides); and a sample comprising sample cells from a patient.

Cells for use in the methods as described above may be collected from a sample from a subject or a donor, and may optionally may be separated from a mixture of cells by techniques that enrich for desired cells, or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

The collected and optionally enriched cell population may be used immediately or may be frozen at liquid nitrogen temperatures and stored, being thawed and capable of being reused. The cells will usually be stored in 20% DMSO, 80% FCS/human sera.

As used herein, the terms “treatment,” “treating,” and the like, refer to administering an agent, or carrying out a procedure for the purposes of obtaining an effect. The effect may be prophylactic in terms of completely or partially preventing a disease or symptom thereof and/or may be therapeutic in terms of effecting a partial or complete cure for a disease and/or symptoms of the disease.

Treating may refer to any indicia of success in the treatment or amelioration or prevention of disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with a disease. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapy (i.e., first therapeutic agent) and a second therapy. When administered in combination, each component can be administered at the same time or sequentially in any order at different points in time. Thus, each component can be administered separately but sufficiently closely in time so as to provide the desired therapeutic effect.

“Concomitant administration” of a therapy with a second therapy means administration at such time that both components will have a therapeutic effect. Such concomitant administration may involve concurrent (i.e. at the same time), prior, or subsequent administration of a therapy with respect to the administration of a second therapy. A person of ordinary skill in the art would have no difficulty determining the appropriate timing, sequence and dosages of administration for particular drugs and compositions.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize the growth and spread of fatigue syndrome. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen). In some embodiments treatment is pharmacologic, for example including administration of an effective dose of therapeutic agents that modify ROS pathways, such as metformin, etc. In some embodiments a therapeutic agent is identified by a screening method disclosed herein.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).)

The term “flow cytometry” as used herein refers to a method and a process whereby cells within a sample can be detected and identified when transversing past a detector within an apparatus containing a detecting source and a flowing apparatus, e.g. FACS and mass cytometry. FACS rapidly analyzes single cells or particles as they flow past single or multiple lasers while suspended in a buffered salt-based solution. Each particle is analyzed for visible light scatter and one or multiple fluorescence parameters. Visible light scatter is measured in two different directions, the forward direction (Forward Scatter or FSC) which can indicate the relative size of the cell and at 90° (Side Scatter or SSC) which indicates the internal complexity or granularity of the cell. Light scatter is independent of fluorescence. Samples are prepared for fluorescence measurement through transfection and expression of fluorescent dyes. Traditional flow cytometers consist of three systems: fluidics, optics and electronics. The fluidics system consists of sheath fluid (usually a buffered saline solution) that is pressurized to deliver and focus the sample to the laser intercept or interrogation point where the sample is analyzed. The optical system consists of excitation optics (lasers) and collection optics (photomultiplier tubes or PMTs and photodiodes) that generate the visible and fluorescent light signals used to analyze the sample. A series of dichroic filters steer the fluorescent light to specific detectors and bandpass filters determine the wavelengths of light that are read so that each individual fluorochrome can be detected and measured. More specifically, dichroic filters are filters that pass light through that is either shorter or longer in wavelength and reflect the remaining light at an angle. For example, a 450 Dichroic Long Pass filter (DLP) lets light that has a longer wavelength than 450 nm through the filter and bounces the shorter wavelengths of light off at an angle to be sent to another detector. Bandpass filters detect a small window of a specific wavelength of light. For example, a 450/50 bandpass filter passes fluorescent light that has a wavelength of 450 nm+/−25 nm through the filter to be read by the detector. The electronic system converts the signals from the detectors into digital signals that can be read by a computer. Multiple laser systems are common with instruments often having 20 parameters (FSC, SSC and 18 fluorescent detectors).

Oxidative stress results from an imbalance in the production of reactive oxygen species (ROS) and the ability of the cell to scavenge them. ROS react with nucleic acids, proteins and lipids causing cell and tissue damage and can be measured using selective or general indicators. General indicators for the presence of ROS include DCFDA, CellROX reagents; etc.

Dyes for detection of oxidative stress biomarkers in flow cytometry are known and commercially available. For example, reduced glutathione (GSH) is a major thiol bound to proteins. Protein thiols including GSH play an important role in determining the redox status of cells. Therefore, detection of reduced GSH levels is a useful indication of redox potential and a cell's ability to prevent oxidative stress. ThiolTracker; monochlorobimane, monobromobimane, and Grx-1-roGFP, for example, may be used.

Superoxide, peroxyl radical, hydrogen peroxide, hydroxyl radical and peroxynitrite are some examples of ROS that react with nucleic acids, proteins and lipids and result in cell and tissue damage. Certain ROS have been implicated in various human diseases including cancer, cardiovascular disease, neurodegenerative disease and aging. Available dyes include MitoSOX red superoxide indicator; MitoSOX green superoxide indicator; dihydroethidium; DAF-FM Diacetate; and Premo Cellular Hydrogen Peroxide (H2O2) Sensor.

Lipid peroxidation is the oxidative degradation of lipids. Reactive oxygen species are known to be the major initiators of lipid peroxidation and membrane bound polyunsaturated fatty acids like arachidonic acid and linoleic acid are their major targets. The byproducts of lipid peroxidation cause direct damage to cell membranes. They also form protein adducts resulting in cell and tissue damage. Lipid peroxidation is implicated in many human diseases including diabetes and cardiovascular disease. Image-iT lipid peroxidation is suitable for this purpose.

In some embodiments, a biomarker of a fatigue syndrome is increased lymphocyte proliferation in response to stimulus, which may be related to altered oxidative stress. In some such embodiments the lymphocyte proliferation is determine by labeling the a cell sample comprising lymphocytes with a tracer dye, e.g. CellTrace™, CSFE, CMAC, Blue CMF2HC, Violet BMQC, Green CMFDA, Orange CMRA, CM-Dil, CMTPX, Deep Red, etc. The labeled cells are exposed to a stimulus, e.g. anti-CD3, IL-2 and anti-CD28; allogeneic cells; antigen; etc. The level of proliferation can be determined by dye levels, e.g. by flow cytometry, where there is an increase in disease samples relative to a healthy control.

Elemental mass spectrometry-based flow cytometry (mass cytometry) is a method to characterize single cells or particles with elemental metal isotope-labeled binding reagents. Because there are many stable metal isotopes available, and little overlap between measurement channels, dozens of molecules (parameters) can be readily measured. An example of a mass cytometer used to read the metal tags is an inductively-coupled plasma mass spectrometer (ICP-MS). In a typical workflow (similar to fluorescence based cytometry), cells are first incubated with antibodies/affinity binders conjugated to pure isotopes and subsequently the cell suspension is injected as a single cell stream into the mass cytometer. Single cell droplets are generated via nebulization and are carried by an argon gas stream plasma where each single cell is completely atomized and ionized. Thereby generated metal ions are then directed into a time-of-flight (TOF) mass spectrometer and the mass over charge ratio and number of metal ions is measured per cell and thereby the abundance of the target epitope/molecules.

As used herein, the term “elemental analysis” refers to a method by which the presence and/or abundance of elements of a sample are evaluated. “Capacitively coupled plasma” (CCP) means a source of ionization in which a plasma is established by capacitive coupling of radiofrequency energy at atmospheric pressure or at a reduced pressure (typically between 1 and 500 Torr) in a graphite or quartz tube.

“Mass spectrometer” means an instrument for producing ions in a gas and analyzing them according to their mass/charge ratio. “Microwave induced plasma” (MIP) means a source of atomization and ionization in which a plasma is established in an inert gas (typically nitrogen, argon or helium) by the coupling of microwave energy. The frequency of excitation force is in the GHz range. “Glow discharge” (GD) means a source of ionization in which a discharge is established in a low pressure gas (typically between 0.01 and 10 Torr), typically argon, nitrogen or air, by a direct current (or less commonly radiofrequency) potential between electrodes. “Graphite furnace” means a spectrometer system that includes a vaporization and atomization source comprised of a heated graphite tube. Spectroscopic detection of elements within the furnace may be performed by optical absorption or emission, or the sample may be transported from the furnace to a plasma source (e.g. inductively coupled plasma) for excitation and determination by optical or mass spectrometry.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

A “dataset” is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring antibody binding, or other methods of quantitating a signaling response. The phrase also encompasses receiving a set of data, e.g., from a third party that has processed the sample to experimentally determine the dataset.

“Measuring” or “measurement” in the context of the present teachings refers to determining the presence, absence, quantity, amount, or effective amount of a cell population in a clinical or subject-derived sample, including the presence, absence, or concentration levels of such cells, and/or evaluating the values or categorization of a subject's clinical parameters based on a control, e.g. baseline levels of the cell population.

Classification can be made according to predictive modeling methods that set a threshold for determining the probability that a sample belongs to a given class. The probability preferably is at least 50%, or at least 60% or at least 70% or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

Classification is the process of recognizing, understanding, and grouping ideas and objects into preset categories or “sub-populations.” Using pre-categorized training datasets, machine learning programs use a variety of algorithms to classify future datasets into categories. Classification algorithms in machine learning use input training data to predict the likelihood that subsequent data will fall into one of the predetermined categories. An analytic classification process may use any one of a variety of statistical analytic methods to manipulate the quantitative data and provide for classification of the sample. Examples of useful methods include linear discriminant analysis, recursive feature elimination, a prediction analysis of microarray, a logistic regression, a CART algorithm, a FlexTree algorithm, a LART algorithm, a random forest algorithm, a MART algorithm, machine learning algorithms; etc. Using any one of these methods, a protein distribution pattern may be used to generate a predictive model. In the generation of such a model, a dataset comprising control, and fatigue syndrome are used as a training set. A training set will contain data for one or more different distributions of interest. In some embodiments a decision tree is used to order classes on a precise level, for example with a random forest algorithm.

The predictive ability of a model can be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about at least about 0.7, at least about 0.75, at least about 0.8, at least about 0.85, at least about 0.9, or higher.

The raw data may be initially analyzed by measuring the values for each marker, usually in triplicate or in multiple triplicates; and the cells may be clustered into populations, e.g. with flowSOM. The data may be analyzed, for example, raw data may be transformed using standard curves, and the average of triplicate measurements used to calculate the average and standard deviation for each patient. These values may be transformed before being used in the models, e.g. log-transformed, Box-Cox transformed (see Box and Cox (1964) J. Royal Stat. Soc., Series B, 26:211-246), etc. The data are then input into a predictive model, which will classify the sample according to the state. The resulting information may be transmitted to a patient or health professional.

The term “specific binding member” as used herein refers to a member of a specific binding pair, i.e. two molecules, usually two different molecules, where one of the molecules through chemical or physical means specifically binds to the other molecule. For the purposes of the present invention, one of the molecules is an analyte as defined above, and generally the specific binding member is labeled for detection of fluorescence or elemental analysis, as known in the art.

The complementary members of a specific binding pair are sometimes referred to as a ligand and receptor; or receptor and counter-receptor. Specific binding indicates that the agent can distinguish a target antigen, or epitope within it, from other non-target antigens. It is specific in the sense that it can be used to detect a target antigen above background noise (“non-specific binding”). For example, a specific binding partner can detect a specific sequence or a topological conformation. A specific sequence can be a defined order of amino acids or a defined chemical moiety (e.g., where an antibody recognizes a phosphotyrosine or a particular carbohydrate configuration, etc.) which occurs in the target antigen. The term “antigen” is issued broadly, to indicate any agent which elicits an immune response in the body. An antigen can have one or more epitopes.

Binding pairs of interest include antigen and antibody specific binding pairs, complementary nucleic acids, peptide-MHC-antigen complexes and T cell receptor pairs, biotin and avidin or streptavidin; carbohydrates and lectins; complementary nucleotide sequences; peptide ligands and receptor; effector and receptor molecules; hormones and hormone binding protein; enzyme cofactors and enzymes; enzyme inhibitors and enzymes; and the like. The specific binding pairs may include analogs, derivatives and fragments of the original specific binding member. For example, an antibody directed to a protein antigen may also recognize peptide fragments, chemically synthesized peptidomimetics, labeled protein, derivatized protein, etc. so long as an epitope is present.

Immunological specific binding pairs include antigens and antigen specific antibodies; and T cell antigen receptors, and their cognate MHC-peptide conjugates. Suitable antigens may be haptens, proteins, peptides, carbohydrates, etc. Recombinant DNA methods or peptide synthesis may be used to produce chimeric, truncated, or single chain analogs of either member of the binding pair, where chimeric proteins may provide mixture(s) or fragment(s) thereof, or a mixture of an antibody and other specific binding members. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

A nucleic acid based binding partner such as an oligonucleotide can be used to recognize and bind DNA or RNA based analytes. The term “polynucleotide” as used herein may refer to peptide nucleic acids, locked nucleic acids, modified nucleic acids, and the like as known in the art. The polynucleotide can be DNA, RNA, LNA or PNA, although it is not so limited. It can also be a combination of one or more of these elements and/or can comprise other nucleic acid mimics.

Binding partners can be primary or secondary. Primary binding partners are those bound to the analyte of interest. Secondary binding partners are those that bind to the primary binding partner.

In one embodiment analysis is performed on a flow cytometer, in which cells are introduced into a fluidic system and introduced into the cytometer one cell at a time. In one embodiment, cells are carried in a liquid suspension and sprayed into a plasma source by means of a nebulizer. In another embodiment, the cells may be hydrodynamically focused one cell at a time through a flow cell using a sheath fluid. In particular embodiments, the cells may be compartmentalized in the flow cell by introduction of an immiscible barrier, e.g., using a gas (e.g., air or nitrogen) or oil, such that the cell is physically separated from other cells that are passing through the flow cell. The cells may be compartmentalized prior to or during introduction of the cell into the flow cell by introducing an immiscible material (e.g., air or oil) into the flow path.

The results of such analysis may be compared to results obtained from reference compounds, concentration curves, controls, etc. The comparison of results is accomplished by the use of suitable deduction protocols, AI systems, statistical comparisons, etc.

In particular embodiments, the method described above may be employed in a multiplex assay in which a heterogeneous population of cells is labeled with a plurality of distinguishably labeled binding agents (e.g., a number of different dyes or antibodies). After the population of cells is labeled, the cells are introduced into the flow cell, and individually analyzed using the method described above, where the viable cells are distinguished from non-viable cells by the presence of platinum derived from the viability reagent.

The analyte distribution pattern may be generated from a cell sample using any convenient protocol. The readout may be a mean, average, median or the variance or other statistically or mathematically-derived value associated with the measurement. The readout information may be further refined by direct comparison with the corresponding reference or control pattern. A pattern may be evaluated on a number of points: to determine if there is a statistically significant change at any point in the data matrix; whether the change is an increase or decrease in prevalence of an isoform; and the like. The absolute values will display a variability that is inherent in live biological systems.

### Methods

Analysis of biological samples, e.g. blood-based samples, obtained from an individual is used to obtain a determination of changes in cellular oxidative stress, which are shown herein to be diagnostic of the presence of a fatigue syndrome in the individual.

The sample can be any suitable type that allows for the analysis of one or more cells, particularly lymphocytes, proteins and metabolites, preferably a blood sample. Samples can be obtained once or multiple times from an individual. The cells can be separated from body samples by red cell lysis, centrifugation, elutriation, density gradient separation, apheresis, affinity selection, panning, FACS, centrifugation with Hypaque, solid supports (magnetic beads, beads in columns, or other surfaces) with attached antibodies, etc.

A profile of biomarkers shown herein to be indicative of a fatigue syndrome is determined by measuring the sample, e.g. single cells in the sample, for presence of specific biomarkers. It is understood that marker levels can exist as a distribution and that a marker used to classify a cell can be a particular point on the distribution but more typically can be a portion of the distribution or combination of different markers in specific mathematical forms. In some embodiments of the invention, different gating strategies can be used in order to analyze a specific cell population in a sample of mixed cell population. These gating strategies can be based on the presence of one or more specific surface markers. The following gate can differentiate between dead cells and live cells and the subsequent gating of live cells classifies them into, e.g. myeloid blasts, monocytes and lymphocytes. A clear comparison can be carried out by using two-dimensional contour plot representations, two-dimensional dot plot representations, and/or histograms.

Samples may be obtained at one or more time points. Where a sample at a single time point is used, comparison is made to a reference “base line” level for the feature, which may be obtained from a training set data as disclosed herein.

In some embodiment, the methods of the invention include the use of liquid handling components. The liquid handling systems can include robotic systems comprising any number of components. In addition, any or all of the steps outlined herein can be automated; thus, for example, the systems can be completely or partially automated. As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a mass cyometer; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this can be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

### Drug Screening Assays

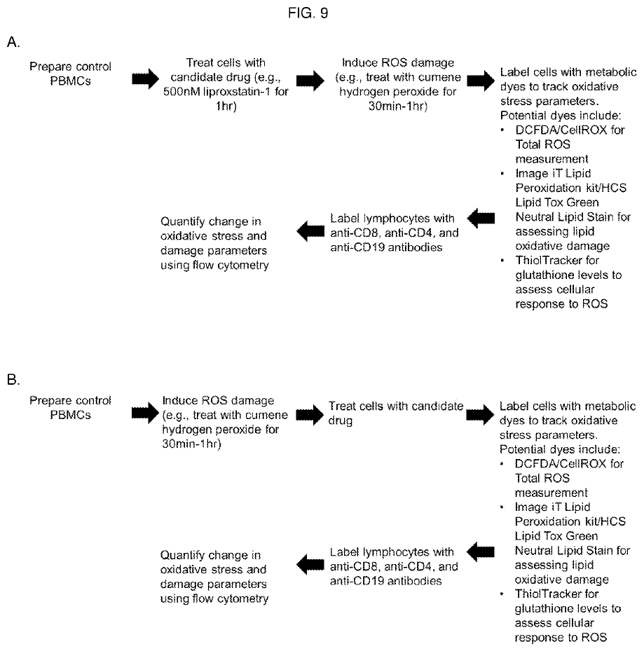

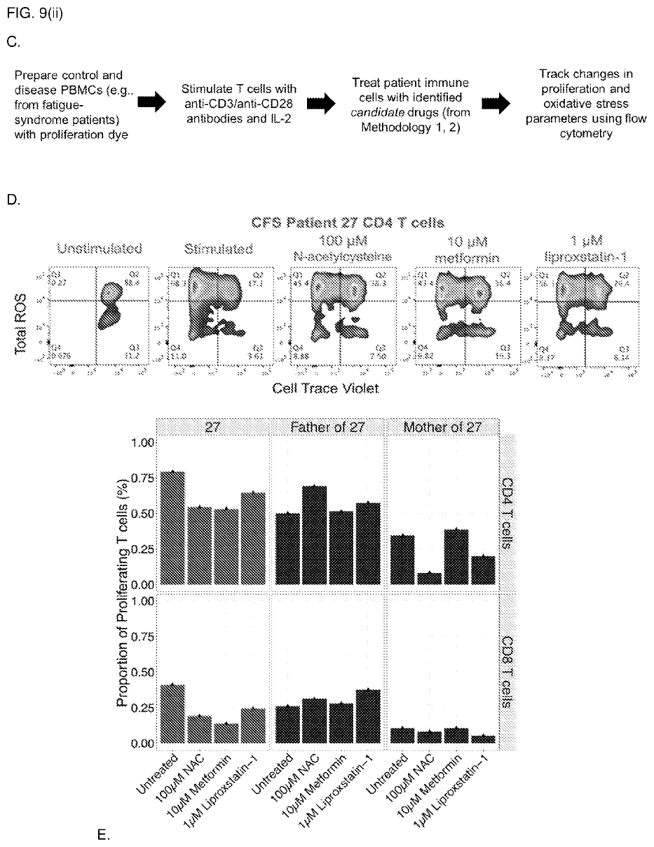

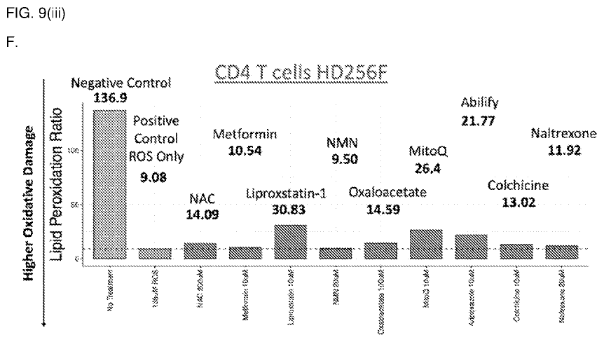

In an embodiment, methods are provided for screening candidate agents for treatment of a fatigue syndrome, for example as shown in FIG. 9. A screening assay of interest utilizes cell-based assays for determining an effect on lymphocyte proliferative and oxidation stress parameters. Cells may be initially tested, for example, to screen candidate agents for ROS-protective, or ROS-reversing effects. In such an assay, a cell population comprising lymphoid cells is exposed to a candidate agent before or exposure to an agent that induces ROS damage, for a period of time sufficient to observe an effect, e.g. from about 1 to 5 days. The cells may be an in vitro cultured cell population, e.g. a lymphoid cell line; primary cells, e.g. PBMC; etc.

The effect of the candidate agent may be monitored by at least one, two, three, four, five, or all of (a) determination of ROS by flow cytometry; (b) determination of the ratio of mitochondrial Ca++ to SOD2 mean fluorescence intensity by flow cytometry; (c) determination of the maximum glutathione (GSH) level by flow cytometry; (d) analyzing by flow cytometry the level of glutathione peroxidase 4 (GPX4); (e) determination of lipid peroxides; (f) determination of lipid droplets; and (g) lymphocyte proliferation in response to stimuli.

In some embodiments a candidate agent is an antioxidant. The accumulation of free radicals can lead to oxidative stress, damaging cells and contributing to the development of chronic conditions. Antioxidant medications may work by scavenging free radicals, reducing oxidative stress, and protecting cells from damage. Classes of antioxidants include, without limitation, vitamins, such as Vitamin E (tocopherols and tocotrienols, and Vitamin C (ascorbic acid); enzymes such as superoxide dismutase (SOD), and catalase; coenzymes such as coenzyme Q10 (CoQ10); minerals such as selenium and zinc; phytochemicals such as flavonoids, and polyphenols; synthetic antioxidants such as N-acetylcysteine (NAC), alpha-lipoic acid (ALA), metformin, etc.; carotenoids such as beta-carotene; and the like.

Candidate agents with ROS-protective or ROS-reversing activity may be further tested on a population of lymphocytes from an individual with a fatigue disorder in an assay as described above, to determine the effectiveness in modifying oxidative stress and proliferative parameters. Agents with activity can be used in treatment of individuals with a fatigue disorder.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

In some embodiments, candidate agents are known drugs or compounds, for example exemplified by the panel of drugs tested in FIG. 9F: metformin; liproxstatin-1; NMN, oxaloacetate; MitoQ; Abilify; colchicine; naltrexone; etc. Many candidate compounds are available for this purpose.

Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference.

Test compounds include all of the classes of molecules described above, and may further comprise samples of unknown content, for example complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1.mu.l to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of test cells, usually in conjunction with cells lacking the agent. The change in parameter readout in response to the agent is measured, desirably normalized, and the result may then be evaluated by comparison to reference responses, e.g. include basal readouts in the presence and absence of the agent and/or oxidative damage, results obtained with other agents, which may or may not include known modulators of known pathways, etc.

Candidate agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected markers. For measuring the amount of a molecule that is present, a convenient method is to label a molecule with a detectable moiety, which may be fluorescent, luminescent, radioactive, enzymatically active, etc. Fluorescent moieties are readily available for labeling virtually any biomolecule, structure, or cell type. Immunofluorescent moieties can be directed to bind not only to specific proteins but also specific conformations, cleavage products, or site modifications like phosphorylation.

The use of high affinity antibody binding and/or structural linkage during labeling provides a method of gating on lymphocytes in a mixed cell population, for example using anti-CD3, anti-CD4, anti-CD8, anti-CD19, are in common use. An abundance of useful dyes are now commercially available. These are available from many sources, including Sigma Chemical Company (St. Louis Mo.) and Molecular Probes (Handbook of Fluorescent Probes and Research Chemicals, Seventh Edition, Molecular Probes, Eugene Oreg.). Other fluorescent sensors have been designed to report on biological activities or environmental changes, e.g. pH, reactive oxygen species, lipids and lipid droplets, calcium concentration, electrical potential, proximity to other probes, etc. Methods of interest include calcium flux, nucleotide incorporation, quantitative PAGE (proteomics), etc.

Multiple fluorescent labels can be used on the same sample and individually detected quantitatively, permitting measurement of multiple cellular responses simultaneously. Many quantitative techniques have been developed to harness the unique properties of fluorescence including: direct fluorescence measurements, fluorescence resonance energy transfer (FRET), fluorescence polarization or anisotropy (FP), time resolved fluorescence (TRF), fluorescence lifetime measurements (FLM), fluorescence correlation spectroscopy (FCS), and fluorescence photobleaching recovery (FPR) (Handbook of Fluorescent Probes and Research Chemicals, Seventh Edition, Molecular Probes, Eugene Oreg.).

Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. These techniques utilize specific antibodies as reporter molecules, which are particularly useful due to their high degree of specificity for attaching to a single molecular target. Cell readouts for proteins and other cell determinants can be obtained using fluorescent or otherwise tagged reporter molecules. Cell based ELISA or related non-enzymatic or fluorescence-based methods enable measurement of cell surface parameters and secreted parameters. Capture ELISA and related non-enzymatic methods usually employ two specific antibodies or reporter molecules and are useful for measuring parameters in solution. Flow cytometry methods are useful for measuring cell surface and intracellular parameters, as well as shape change and granularity and for analyses of beads used as antibody- or probe-linked reagents. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these.

In some embodiments flow cytometry is used to quantitate parameters such (a) determination of ROS by flow cytometry; (b) determination of the ratio of mitochondrial Ca++ to SOD2 mean fluorescence intensity by flow cytometry; (c) determination of the maximum glutathione (GSH) level by flow cytometry; (d) analyzing by flow cytometry the level of glutathione peroxidase 4 (GPX4); (e) determination of lipid peroxides; (f) determination of lipid droplets; and (g) lymphocyte proliferation in response to stimuli. The readouts of selected parameters are capable of being read simultaneously, or in sequence during a single analysis.

The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26 (1): 112-225; Kawamoto et al. (1999) Genome Res 9 (12): 1305-12; and Chen et al. (1998) Genomics 51 (3): 313-24, for examples.

In some embodiments, candidate agents with ROS-protective or ROS-reversing activity are further tested on a population of lymphocytes from an individual with a fatigue disorder to determine clinical potential. For example, samples of PBMLs from control and fatigue syndrome patients and obtained, and optionally labeled with a proliferation dye. The cells are stimulated, e.g. with anti-CD3, anti-CD28 and IL-2; or with allogeneic cells; antigens of interest; etc. The cells are then contacted with a candidate agent for a period of time sufficient to determine an effect, e.g. from about 1-5 days. Changes on oxidative stress markers, and/or lymphocyte proliferation may be monitored by any convenient method, including flow cytometry. Agents that show activity may be further studied in animal models and clinical trials for treatment of fatigue syndrome patients.

### Therapeutic Methods

In some embodiments, following immune cell profiling for oxidative stress and a determination that a fatigue syndrome is present, the individual is treated to ameliorate, diminish, or actively treat the syndrome. Such treatment may prevent progression or reduce severity of the fatigue syndrome. In some embodiments treatment is pharmacologic, for example including administration of therapeutic agents that modify ROS pathways. In some embodiments a therapeutic agent is identified by a screening method disclosed herein. In other embodiments treatment comprises diet, physical and/or occupational therapy. In some embodiments treatment comprises clinical trial enrollment, where individuals can be stratified by likelihood of a fatigue syndrome diagnosis. The oxidative stress markers can be employed as endpoints for studying drug effects in these conditions, where therapies aim to relieve patient fatigue by mitigating existing oxidative damage and/or restoring the balance between oxidative stress/anti-oxidants for improved function of the individuals.

Determining a therapeutically or prophylactically effective amount of an agent can be done based on animal data using routine computational methods. The effective dose may range up to about 30 mg/kg, up to about 20 mg/kg, up to about 10 mg/kg, up to about 5 mg/kg; up to about 1 mg/kg, up to about 0.5 mg/kg; up to about 0.1 mg/kg; up to about 0.05 mg/kg; where the dose may vary with the specific antibody and recipient. The therapeutic dose may be at least about 0.01 μg/kg body weight, at least about 0.05 μg/kg body weight; at least about 0.1 μg/kg body weight, at least about 0.5 μg/kg body weight, at least about 1 μg/kg body weight, at least about 2.5 μg/kg body weight, at least about 5 μg/kg body weight, and not more than about 1000 μg/kg body weight, not more than about 500 μg/kg body weight, not more than about 100 μg/kg body weight. It will be understood by one of skill in the art that such guidelines will be adjusted for the molecular weight of the active agent. The dosage may also be varied for localized administration, e.g. intranasal, inhalation, etc., or for systemic administration, e.g. i.m., i.p., i.v., and the like.

The agent may be administered one or a plurality of days, and in some embodiments is administered daily, every two days, semi-weekly, weekly, etc. for a period of from about 1, about 2, about 3, about 4, about 5, about 6, about 7 or more weeks, up to a chronic maintenance level of dosing. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the drug in the patient.

In certain embodiments, multiple therapeutically effective doses are administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, once every two weeks, once every three weeks, once a month, and so forth. For example, in some embodiments, an antibody is administered once every two to four weeks for an extended period of time, such as for 1, 2, 3, 4, 5, 6, 7, 8, 10, 15, 24 months, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy for one or more weekly or monthly cycles until the desired therapeutic response is achieved. The agents can be administered by any acceptable route of administration as noted herein below.

Administering the compositions can be effected or performed using any of the various methods and delivery systems known to those skilled in the art. The administering can be performed, for example, intravenously, orally, via implant, transmucosally, transdermally, intramuscularly, intrathecally, and subcutaneously. The following delivery systems, which employ a number of routinely used pharmaceutical carriers, are only representative of the many embodiments envisioned for administering compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprolactone.

Oral delivery systems include tablets and capsules. These can contain excipients such as binders (e.g., hydroxypropylmethylcellulose, polyvinyl pyrolidone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, xanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and Jun. 2, 2005 antioxidants (e.g., parabens, vitamins E and C, and ascorbic acid), anti-caking agents, coating agents, and chelating agents (e.g., EDTA).

### Data Analysis

A signature pattern can be generated from a biological sample using any convenient protocol, for example as described below. The readout can be a mean, average, median or the variance or other statistically or mathematically-derived value associated with the measurements. The marker readout information can be further refined by direct comparison with the corresponding reference or control pattern. A population distribution pattern can be evaluated on a number of points: to determine if there is a statistically significant change at any point in the data matrix relative to a reference value; whether the change is an increase or decrease in the population frequency; and the like. The absolute values obtained for each marker under identical conditions will display a variability that is inherent in live biological systems and also reflects the variability inherent between individuals.

Following obtainment of the signature pattern from the sample being assayed, the signature pattern can be compared with a reference or base line profile to make a prognosis regarding the phenotype of the patient from which the sample was obtained/derived.

In certain embodiments, the obtained signature pattern is compared to a single reference/control profile to obtain information regarding the phenotype of the patient being assayed. In yet other embodiments, the obtained signature pattern is compared to two or more different reference/control profiles to obtain more in depth information regarding the phenotype of the patient. For example, the obtained signature pattern can be compared to a positive and negative reference profile to obtain confirmed information regarding whether the patient has the phenotype of interest.

The data can be subjected to non-supervised hierarchical clustering to reveal relationships among profiles. For example, hierarchical clustering can be performed, where the Pearson correlation is employed as the clustering metric. One approach is to consider a patient disease dataset as a “learning sample” in a problem of “supervised learning”. CART is a standard in applications to medicine (Singer (1999) Recursive Partitioning in the Health Sciences, Springer), which can be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

Other methods of analysis that can be used include logistic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as Prediction Analysis of Microarrays (PAM) software, a software “plug-in” for Microsoft Excel, and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome. Several of these methods are based on the “R” software, developed at Stanford University, which provides a statistical framework that is continuously being improved and updated in an ongoing basis.

Other statistical analysis approaches including principle components analysis, recursive partitioning, predictive algorithms, Bayesian networks, random forest, and neural networks.

The analysis and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data can be used for a variety of purposes, such as patient monitoring, initial diagnosis, clinical trial analysis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### Kits

In some embodiments, the invention provides kits for the classification, diagnosis, prognosis, theragnosis, and/or prediction of early fatigue syndrome in a subject. The kit may further comprise a software package for data analysis of the cellular state and its physiological status, which may include reference profiles for comparison with the test profile and comparisons to other analyses as referred to above. The kit may also include instructions for use for any of the above applications.

Kits provided by the invention may comprise one or more of the affinity reagents described herein. A kit may also include other reagents that are useful in the invention, such as dyes, modulators, fixatives, containers, plates, buffers, therapeutic agents, instructions, and the like.

Kits provided by the invention can comprise one or more labeling elements. Non-limiting examples of labeling elements include small molecule fluorophores, proteinaceous fluorophores, radioisotopes, enzymes, antibodies, chemiluminescent molecules, biotin, streptavidin, digoxigenin, chromogenic dyes, luminescent dyes, phosphorous dyes, luciferase, magnetic particles, beta-galactosidase, amino groups, carboxy groups, maleimide groups, oxo groups and thiol groups, quantum dots, chelated or caged lanthanides, isotope tags, radiodense tags, electron-dense tags, radioactive isotopes, paramagnetic particles, agarose particles, mass tags, e-tags, nanoparticles, and vesicle tags.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

### Reports

In some embodiments, providing an evaluation of a subject for a classification, diagnosis, prognosis, theragnosis, and/or prediction of early fatigue syndrome includes generating a written report that includes the artisan's assessment of the subject's state of health, including, for example, a “diagnosis assessment”, of the subject's prognosis, i.e. a “prognosis assessment”, and/or of possible treatment regimens, i.e. a “treatment assessment”. Thus, a subject method may further include a step of generating or outputting a report providing the results of an assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a diagnosis assessment, a prognosis assessment, and/or a treatment assessment and its results. A subject report can be completely or partially electronically generated. A subject report includes at least a diagnosis assessment, and/or a suggested course of treatment to be followed. A subject report can further include one or more of: 1) information regarding the testing facility; 2) service provider information; 3) subject data; 4) sample data; 5) an assessment report, which can include various information including: a) test data, where test data can include an analysis of cellular signaling responses to activation, b) reference values employed, if any.

The report may include information about the testing facility, which information is relevant to the hospital, clinic, or laboratory in which sample gathering and/or data generation was conducted. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the diagnosis, prognosis, or treatment assessment) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the susceptibility prediction and, if different from the ordering physician, the name of a staff physician who is responsible for the subject's care (e.g., primary care physician).

The report may include a sample data section, which may provide information about the biological sample analyzed, such as the source of biological sample obtained from the subject (e.g. blood, type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prognosis of the likelihood that the patient will develop preeclampsia. The interpretive report can include, for example, results of the analysis, methods used to calculate the analysis, and interpretation, i.e. prognosis. The assessment portion of the report can optionally also include a Recommendation(s).

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., a diagnosis, a prognosis, or a prediction of responsiveness to a therapy).

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Oxidative Stress is a Shared Characteristic of ME/CFS and Long COVID

More than 65 million individuals worldwide are estimated to have Long COVID (LC), a complex multisystemic condition, wherein patients of all ages report fatigue, post-exertional malaise, and other symptoms resembling myalgic encephalomyelitis/chronic fatigue syndrome (ME/CFS). With no current treatments or reliable diagnostic markers, there is an urgent need to define the molecular underpinnings of these conditions. By studying bioenergetic characteristics of peripheral blood lymphocytes in over 16 healthy controls, 15 ME/CFS, and 15 LC, we find both ME/CFS and LC donors exhibit signs of elevated oxidative stress, relative to healthy controls, especially in the memory subset. Using a combination of flow cytometry, bulk RNA-seq analysis, mass spectrometry, and systems chemistry analysis, we also observed aberrations in ROS clearance pathways including elevated glutathione levels, decreases in mitochondrial superoxide dismutase levels, and glutathione peroxidase 4 mediated lipid oxidative damage. Critically, these changes in redox pathways show striking gender-specific trends. While females diagnosed with ME/CFS exhibit higher total ROS and mitochondrial calcium levels, males with an ME/CFS diagnosis have normal ROS levels, but larger changes in lipid oxidative damage. Further analyses show that higher ROS levels correlates with hyperproliferation of T cells, consistent with the known role of elevated ROS levels in the initiation of proliferation. This hyperproliferation of T cells can be attenuated by metformin. Thus, we report that both ME-CFS and LC are mechanistically related and can be diagnosed with quantitative blood cell measurements. We also show that effective, patient tailored drugs might be discovered using standard lymphocyte stimulation assays.

Here we identify shared molecular signatures between ME/CFS and LC donors versus healthy controls, specifically signs of oxidative stress. We focused on immune cell bioenergetics based on several lines of evidence. Multiple studies have found signs of inflammation in LC and CFS patients, including higher serum cytokine levels that are associated with patient-reported fatigue severity and changes in both lymphocyte and monocyte cell populations among LC donors. These studies have shown that the immune system may play a role in ME/CFS/LC pathogenesis and clinical symptoms.

Fatigue is the principal hallmark of ME/CFS and also one of the most common symptoms among LC patients. Since fatigue implies over and/or misallocation of cellular energy on disease processes, studying metabolism can meaningfully identify aberrations in how cells use and produce energy. Based on plasma metabolomic and proteomic studies, multiple metabolic pathway aberrations have been detected in LC and ME/CFS samples, including changes in serotonin and tryptophan metabolism, cholesterol metabolism, dysfunctional gut microbial butyrate biosynthesis pathways, oxygen transport to tissues, and deficient mitochondrial fatty acid oxidation and ATP metabolism.

We also focused on cells of the immune system because they are a critical consumer of host energy and regulator of host metabolism. Specifically, the immune system accounts for 15-20% daily energy expenditure in humans. This energy is thought to increase to 25%, during a serious infection, highlighting the significant energy expenditure required for maintenance, activation, and proliferation of immune cells. Among the intracellular pathways in immune cells, mitochondrial metabolism is especially relevant. In addition to its central role in generating over 95% of a cell's energy through ATP, changes in mitochondrial morphology and function accompany lymphocyte differentiation and activation, with mitochondria driving cytokine production, thereby linking energy metabolic deficits in immune cells with chronic inflammation. Along these lines, studies have hypothesized and identified signatures of mitochondrial dysfunction among CFS and LC patients, including down-regulation of host mitochondrial genes even after COVID-19 recovery, altered mitochondrial morphology in ME/CFS donor T cells, decreases in mitochondrial membrane potential among recovered COVID-19 subjects' lymphocytes, and redox dysregulation in both ME/CFS and COVID-19.

Reactive oxygen species (ROS) are at the nexus of chronic inflammation and metabolic regulation, with its critical role in driving mitochondrial oxidative phosphorylation, inflammatory cytokine activation, and tissue damage (characteristics of COVID-19 recovery). Based on the separate lines of evidence implicating chronic inflammation and metabolic dysregulation in ME/CFS and LC, we broadly profiled mitochondrial bioenergetic changes, specifically redox parameters, in lymphocytes from peripheral blood mononuclear cells (PBMCs) of 16 healthy controls, 15 ME/CFS, and 15 LC donors. From several measurements capturing ROS levels, oxidative damage, and changes in mitochondrial redox pathways, our findings identify elevated oxidative stress among lymphocytes is a shared molecular feature of ME/CFS and LC, associated with specific functional proliferation defects. Particularly striking was that only female patients had elevated ROS levels in ME-CFS and LC, together with the hyperproliferation of lymphocytes after stimulation in culture, whereas both sexes showed evidence of elevated levels of reduced glutathione and lipid oxidative damage. Thus, while there are major phenotypic differences between the sexes, they converge on evidence of oxidative stress and mitochondrial damage, which may lead to this type of immune system dysfunction acting as an “energy sink” analogous to a severe infection, leading to the symptoms characteristic of these diseases.

### Results

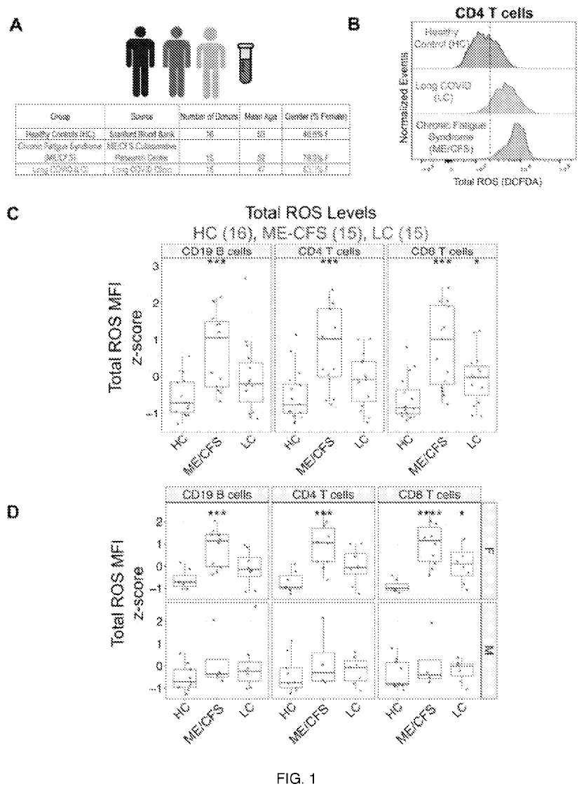

Elevated reactive oxygen species in ME/CFS and Long COVID donor lymphocytes, compared to healthy controls. To profile bioenergetic parameters, we obtained healthy control, ME/CFS, and LC PBMCs. ME/CFS and LC donor PBMCs from the ME/CFS Collaborative Research Center and the Stanford Post-Acute COVID Syndrome clinic, respectively. Healthy control patient PBMCs were obtained from the Stanford Blood Bank, where donors were screened using a medical history questionnaire. ME/CFS patients, including patients meeting the National Academy of Medicine (NAM) ME/CFS criteria before and after the start of the COVID-19 pandemic, were diagnosed by a physician using the National Academy of Medicine and Canadian Consensus criteria. LC donors were diagnosed using the combination of the Center for Disease Control criteria for “Long COVID or post-COVID conditions” and a symptom and functional status questionnaire. Together, these measures capture whether patients report new symptoms four or more weeks after COVID infection and incorporates the Post-COVID 19 Functional Status (PCFS) Scale. Additional details related to patient characteristics in each group are included in the methods section. FIG. 1A shows that the mean ages are balanced across all three cohorts, and the proportion of females is roughly half among healthy controls and LC donors, with over 75% of ME/CFS donors being female. This distribution reflects the higher incidence of ME/CFS in women compared to men.

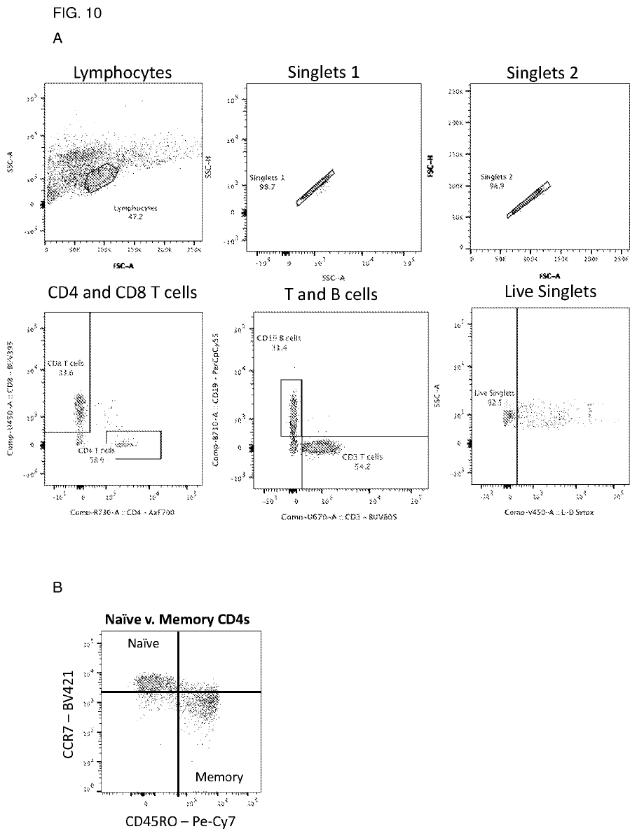

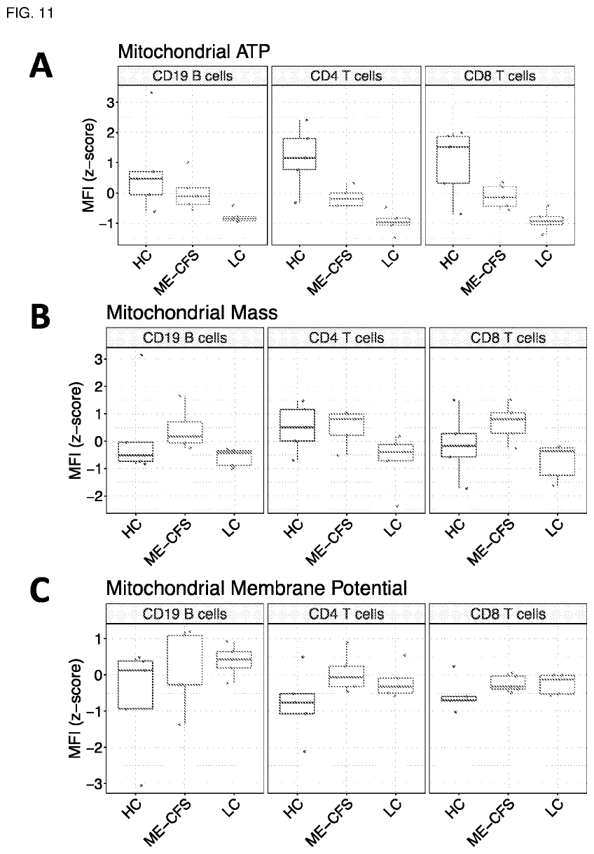

Using flow cytometry, we compared mitochondrial bioenergetic parameters in CD19 B cells as well as CD4 and CD8 T cell lymphocyte populations. Sample gating strategy for identifying these lymphocyte populations is shown in FIG. 10A. Although we did not identify any differences between healthy controls and ME/CFS or LC donors in mitochondrial mass or membrane potential (FIG. 11), decreases in mitochondrial ATP levels were observed across ME/CFS (1.6× decrease on average; HC v. ME-CFS two-sided t-test CD4 T cells p=0.0458, CD8 T cells p=0.0974, CD19 B cells p=0.356) and LC donors (2.8× decrease on average; HC v. LC two-sided t-test CD4 T cells p=0.0076, CD8 T cells p=0.0182, CD19 B cells p=0.0842), compared to healthy controls (FIG. 11). Our findings are consistent with a published study showing oxidative phosphorylation defects among PBMCs in CFS patients, compared to healthy controls. As the decrease in mitochondrial ATP levels implies functional mitochondrial deficits and mitochondria significantly contribute to cellular ROS production through oxidative phosphorylation, we measured total ROS levels. Flow cytometric staining using 2′,7′-dichlorofluorescein diacetate (DCFDA) identified elevated reactive oxygen species (ROS) levels among lymphocytes in ME/CFS and LC donors, compared to healthy controls (FIG. 1B, 1C). DCFDA is hydrolyzed intracellularly and oxidized by ROS, including hydroxyl radicals, hydrogen peroxide derived compounds, to form 2′,7′-dichlorofluorescein (DCF). The formation of DCF is associated with increased fluorescence, which can be detected through flow cytometry. Representative flow cytometric plots from HC, LC, and ME/CFS donors are shown in FIG. 1B. Comparisons of DCF median fluorescence intensity (MFI) measurements between groups ROS (FIG. 1C) indicates statistically significant increases in both LC (n=15) and ME/CFS (n=15) donor lymphocytes, compared to healthy controls (n=16) (ME/CFS CD4T p=5.60*10⁻⁴, CD8T p=1.91*10⁻⁴, CD19B p=3.04*10⁻⁴; LC CD4 T cells p=0.104, CD8 T cells p=0.021, CD19 B cells p=0.073).

As our data showed higher ROS levels among ME/CFS donors compared to LC patients, we compared donors according to their clinical symptoms, specifically if they met the ME/CFS NAM diagnostic criteria (FIG. 1D). As roughly half the patients for LC meet the diagnostic criteria for ME/CFS, we grouped ME/CFS and LC donors, who meet the NAM criteria (‘ME/CFS+’ in FIG. 1D) and compared ROS levels with healthy controls and LC donors, who do not meet the NAM criteria (‘ME/CFS−’ in FIG. 1D). Comparison of MFI ROS levels between groups showed that lymphocytes from ME/CFS-positive cohort presented with statistically significant higher ROS levels (CD19 B cells p=3.3*10⁻⁴, CD4 T cells p=5.1*10⁻⁵, CD8 T cells p=3.2*10⁻⁶), compared to donors who are ME/CFS negative.

Closer examination of the distribution of MFI ROS values among ME/CFS donors (FIG. 1C, 1D) revealed a bimodal distribution, with 8/15 donors (FIG. 1C) exhibiting >2-4× higher ROS levels compared to healthy controls. We found this bimodal distribution could be partially explained by patient's gender, where total ROS levels appear to be uniquely elevated in female ME/CFS+donors (FIG. 1E). Among females (top row, FIG. 1E), total ROS levels are significantly elevated in ME/CFS+donors, compared to ME/CFS-donors (CD19 B cells p=1.6*10⁻³, CD4 T cells p=4.9*10⁻⁴, CD8 T cells p=6.2*10⁻⁶). No differences in ROS levels are found between males with and without ME/CFS (FIG. 1E, bottom row) (CD19 B cells p=0.8, CD4T p=0.66, CD8T p=0.71). Notably, our data offers one potential explanation to account for several epidemiological studies that compare post-COVID 19 and ME/CFS symptoms incidence between males and females. These studies estimate that ME/CFS is three to four times more common in women than men, with significantly higher likelihood for females to develop post COVID-19 symptoms (e.g., 3.3× odds ratio estimate for LC in females v. males).

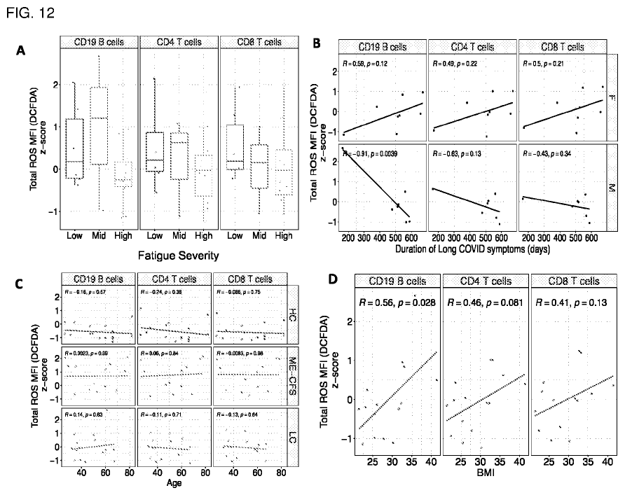

Additionally, The Post-COVID 19 Functional Status (PCFS) Scale and Bell disability score were used to assess fatigue severity. These patient-reported fatigue measures capture a patient's functional status, with a fully functional patient having no symptoms at rest and able to work full-time and an extremely sick patient unable to work, bedridden, and experiencing severe fatigue symptoms on a continuous basis. We did not detect any association between LC/CFS patient-reported fatigue severity and total ROS levels (FIG. 12A), irrespective of gender.

Since the duration of symptoms could be pinpointed for LC patients, we also evaluated whether DCF MFI levels in LC patients are associated with duration of LC symptoms. Although we did not detect a significant association (FIG. 12B), we found that female LC subjects consistently showed a weak positive association and males showed a weak negative association between symptom duration and total ROS levels across lymphocyte populations (females CD19B R=0.59 p=0.12, CD4T R=0.49 p=0.22, CD8T R=0.5 p=0.21; males CD19B R=−0.91 p=0.0039, CD4T R=−0.63 p=0.13, CD8T R=−0.43 p=0.34). These divergent trends in ROS levels suggests that the LC and ME/CFS disease pathogenesis is also distinct between males and females, specifically the mechanisms underlying resolution of sustained oxidative stress.

While we observed no significant association between ROS levels and age (FIG. 12C), we did identify a positive correlation among LC donors between BMI and total ROS levels (CD19 B cells R=0.56, p=0.028; CD4 T cells R=0.46, p=0.081; R=0.14, p=0.13) (FIG. 12D). As BMI has also been positively correlated with the incidence of post-COVID 19 symptoms, our findings suggest that differences in oxidative stress tracks with clinical heterogeneity in ME/CFS and LC. Moreover, these results highlight that elevated ROS levels in lymphocytes are a more sensitive molecular marker of ME/CFS and LC in females, particularly those with higher BMI.

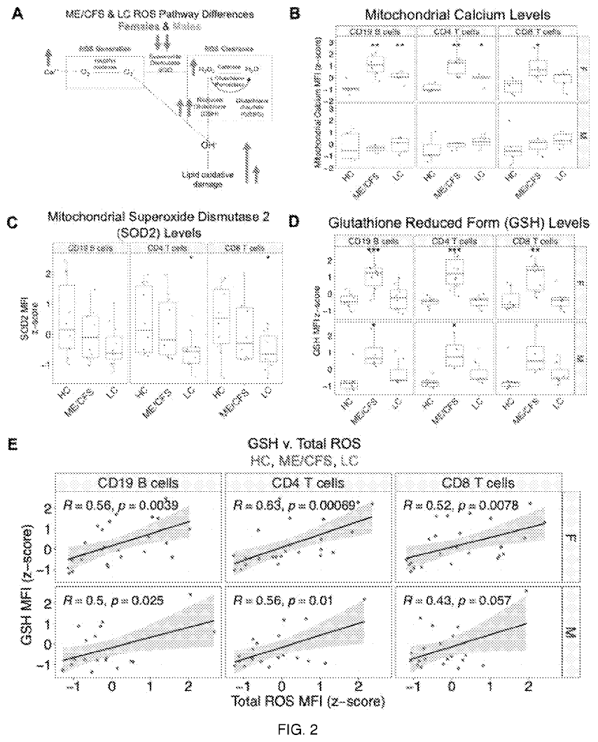

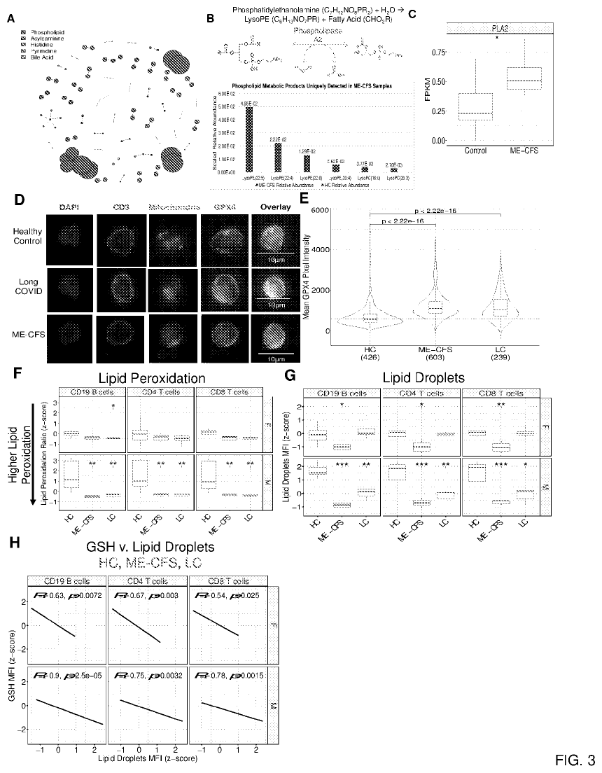

Changes in ROS levels are accompanied by alterations in oxidative stress pathways. Based on our results showing differences in ROS levels, we specifically investigated how ROS pathways are altered in ME/CFS and LC donors, including those related to anti-oxidant clearance and subsequent oxidative damage. FIG. 2A summarizes some of the critical ROS generation and clearance pathways, for which we conducted several measurements to evaluate differences between HC and LC/ME/CFS groups. As depicted in FIG. 2A, intracellular ROS levels are mediated by the balance between ROS-generation and antioxidant processes. In terms of ROS-generation pathways, calcium can drive the production of mitochondrial ROS through electron transport chain activity coupled to NADPH oxidases. In contrast, to regulate mitochondrial ROS levels (e.g., superoxide (O₂⁻)), superoxide dismutase 2 (SOD2) converts superoxide to hydrogen peroxide (H₂O₂). Hydrogen peroxide can be subsequently transformed to H₂O and O₂ through catalase and glutathione peroxidase, which is coupled to oxidation of glutathione (GSH). Moreover, FIG. 2A summarizes the measured gender-specific differences in ROS generation, clearance, and down-stream pathways, where ME/CFS females show elevated mitochondrial calcium and ROS levels, whereas ME/CFS males show larger changes in lipid oxidative damage, compared to healthy counterparts.

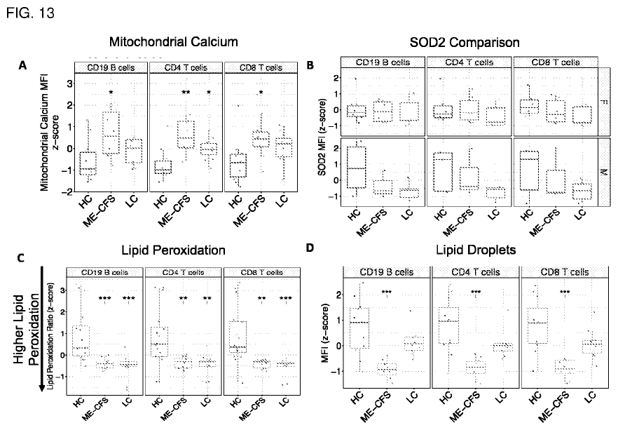

From flow cytometry analysis, we compared mitochondrial Ca²⁺ levels using Rhod-2 AM, a high affinity calcium indicator that primarily localizes in mitochondria. Compared to healthy controls, lymphocytes from ME/CFS and LC donors have 1.67× and 1.32× respectively higher calcium levels, with CD4 T cells showing statistically significant elevations in ME/CFS and LC groups compared to healthy controls (ME/CFS CD19 p=0.014, CD4 p=0.003, CD8 p=0.03; LC CD19 p=0.221, CD4 p=0.023, CD8=0.132) (FIG. 13A). Notably, the mitochondrial calcium levels were uniquely elevated in female ME/CFS and LC donor lymphocytes, compared to healthy controls (FIG. 2B) (females: ME/CFS v. HC CD19B p=0.00238, CD4T p=0.0063, CD8T p=0.0137; ME/CFS v. LC CD19B p=0.0097, CD4T p=0.0172, CD8T p=0.240; males: ME/CFS v. HC CD19B p=0.791, CD4T p=0.514, CD8T p=0.825; LC v. HC CD19B p=0.851, CD4T p=0.241, CD8T p=0.358). To protect against mitochondrial oxidative stress, superoxide dismutase 2 (SOD2) enables conversion of electron transport chain derived superoxide to hydrogen peroxide. Therefore, we also compared SOD2 levels (FIG. 2C) between healthy controls and ME/CFS/LC subjects. Our comparison found that LC donor CD4 and CD8 T cells have significantly lower SOD2 levels, compared to healthy controls (ME/CFS CD19 p=0.326, CD4 p=0.63, CD8 p=0.38; LC CD19 p=0.073, CD4 p=0.023, CD8 p=0.023). These differences in SOD2 levels appeared to be similar across both males and females (FIG. 13B).

Combining these differences to capture the balance between mitochondrial ROS drivers and anti-oxidant pathways, we find the ratio of Ca²⁺ to SOD2 MFI levels is highly elevated in both the ME/CFS and LC donors, compared to healthy controls. The mitochondrial calcium to SOD2 ratio is elevated by 1.67× in LC donors and 1.75× in ME/CFS donors across lymphocytes, compared to healthy controls (ME/CFS: CD19 p=0.011, CD4 p=0.005, CD8 p=0.025; LC: CD19 p=0.05, CD4 p=0.006, CD8 p=0.025). Importantly, these results further show that mitochondrial dysfunction is shared between ME/CFS and LC donors.

To avoid cellular oxidative damage, glutathione can act as a ROS scavenger, where the reduced form of glutathione (GSH) reduces H2O2 to restore cellular redox balance. Glutathione acts as an important reducing agent in several cellular redox reactions, including those catalyzed by glutathione reductase, glutathione peroxidase, catalase. The redox balancing reaction between hydrogen peroxide and glutathione is shown in FIG. 2A and can be written as follows: H2O2+2GSH [symbol] 2H2O+GSSG. Based on this reaction and the hypothesis that endogenous glutathione deficiency is a driver of severe COVID-19 disease, we compared GSH levels using flow cytometry between HC, LC, and ME/CFS donors. Surprisingly, our comparisons found significantly higher GSH levels in ME/CFS donors, across all lymphocyte populations (HC v. ME/CFS CD19 B cells p=2.0*10⁻⁶, CD4 T cells p=5.7*10⁻⁷, CD8 T cells p=1.7*10⁻⁵; HC v. LC CD19 B cells p=0.14, CD4 T cells p=0.064, CD8 T cells p=0.38). As we identified higher ROS levels only in females (FIG. 1D), we also compared glutathione levels in ME/CFS and LC males and females. Our comparison (FIG. 2D) found both ME/CFS males and females exhibit higher GSH levels, compared to controls of the same gender (females ME/CFS v. HC CD19 B cells p=0.00048, CD4 T cells p=0.00014, CD8 T cells p=0.00159; LC v. HC CD19B cells p=0.580, CD4 T cells p=0.827, CD8T cells p=0.958; males ME/CFS v. HC CD19 B cells p=0.0327, CD4 T cells p=0.0354, CD8 T cells p=0.0894; LC v. HC CD19 B cells p=0.196, CD4 T cells p=0.093, CD8 T cells p=0.278). Since DCFDA inference of total ROS (FIG. 1C) can capture cellular hydrogen peroxide levels38, we also evaluated the association between total ROS and GSH measurements (FIG. 2E). As shown in FIG. 2E, we noted a significant positive correlation between total ROS and GSH levels across all lymphocyte populations (females CD19 B cells R=0.56, p=0.0039, CD4 T cells R=0.63 p=6.9*10-4; CD8 T cells R=0.52 p=7.8*10⁻³; males CD19 B cells R=0.5 p=0.025, CD4 T cells R=0.56 p=0.01, CD8 T cells R=0.43 p=0.057). Importantly, although ME/CFS and LC female lymphocytes uniquely exhibit higher total ROS levels, higher GSH levels across both males and females highlight that response to oxidative stress is a common feature in ME/CFS and LC lymphocytes across both sexes.

Elevated oxidative stress in ME/CFS and LC donors is associated with lipid damage. We also probed the likely down-stream metabolic consequences of oxidative stress, specifically whether there was evidence of oxidative damage in known pathways.

To study differences in these pathways, we separately pooled 200,000 sorted CD3+ T cells from both healthy controls and ME/CFS donors and immediately extracted intracellular metabolites. Using hydrophilic interaction liquid chromatography mass spectrometry40, 45,507 unique m/z analytes were detected in samples, of which 747 metabolites were identified using analytical standards. From mass spectrometry data, systems chemistry analysis was conducted using Turium24, a computational program which enabled the mapping of 747 metabolites to 327 pathways. This approach helped identify the top pathways that are increased or uniquely detected in ME/CFS patients, compared to healthy controls (FIG. 3A). In FIG. 3A, each circle corresponds to a specific reaction, up-regulated in ME/CFS donor T cells, compared to healthy controls. The size of the node corresponds to the magnitude of the relative abundance difference between ME/CFS and healthy control samples for the detected metabolite from the mass spectrometry data. We found several metabolic pathway differences, with the most significant corresponding to phospholipid synthesis (FIG. 3A). Specifically, we found that phospholipid metabolites were significantly elevated in ME/CFS samples compared to healthy controls, including lysophosphatidylethanolamine (lysoPE) products (lysoPE (22:5), lysoPE (22:4), lysoPE (22:6), lysoPE (20:4), lysoPE (16:0), lysoPE (20:3)) (FIG. 3B). The reaction to produce these lysoPE products is shown in FIG. 3B, (Phosphotidylethanolamine (C7H12NO8PR2)+H2O→LysoPE (C6H13NO7PR)+CHO2R). These results are consistent with earlier plasma metabolomic studies on ME/CFS that identified significant phospholipid abnormalities.

As the identified reaction products (FIG. 3B) are catalyzed by phospholipase A2 (PLA2), we used RNA-seq data from Bouquet et al. to test for differences in PLA2 transcript expression from 11 sedentary controls and 14 CFS patients whole blood samples. While Bouquet et al. studied the effects of cardiopulmonary exercise on CFS patients, we specifically compared the phospholipase A2 levels (FIG. 3C) at the 7 days post-exercise timepoint, when CFS symptoms returned in patients and lymphocytes were least likely to reflect the effects of exercise. We found that PLA2G4A, a calcium dependent cystolic phospholipase implicated with eicosanoid metabolism, has significantly higher transcript levels in ME/CFS donors compared to controls (p=0.044, two-sided t-test) at day 7 of this study (FIG. 3C). These findings provide an external validation for phospholipid synthesis dysregulation in ME/CFS donors.

Lipid peroxidation of fatty acyl groups occurs predominantly in membrane phospholipids, where lipid peroxidation promotes the formation of several lipid byproducts including lysophospholipids. Based on the differences in phospholipid synthesis and changes in glutathione levels, we evaluated whether ME/CFS and LC donors exhibited signs of lipid oxidative damage, specifically lipid peroxidation.

The conversion of lipid peroxides to lipid alcohols is catalyzed by glutathione peroxidase 4 (GPX4), which protects lipids from ROS damage. Therefore, we conducted immunofluorescence staining of GPX4 (FIG. 3D), across CD3 T cells from a single HC, LC, and ME/CFS donor. Comparison of GPX4 mean pixel intensity from immunofluorescence staining across 426 HC, 603 ME/CFS, and 239 LC CD3 T cells shows that LC and ME/CFS donor lymphocytes have higher (1.77× and 1.9×, respectively) GPX4 levels (FIG. 3E), when compared to T cells from healthy controls (p<2.22*10⁻¹⁶ for both ME/CFS and LC v. HC comparisons, two-sided t-test). Next, using flow cytometry, we measured lipid peroxide levels in HC, ME/CFS, and LC donor lymphocytes, using a ratiometric lipid peroxidation sensor. Our measurements found substantially higher lipid peroxide levels across all lymphocytes in both ME/CFS and LC donors, compared to healthy controls (FIG. 13C), lower y-axis values correspond to higher lipid peroxidation) (HC v. ME/CFS CD19 B cells p=0.000995, CD4 T cells p=0.00556, CD8 T cells p=0.00155; HC v. LC CD19 B cells p=0.000528, CD4 T cells p=0.00281, CD8 T cells p=0.000988). In particular, both ME/CFS and LC males and females presented with similar levels of lipid peroxides (FIG. 3F). However, the differences between controls and ME/CFS/LC donors in lipid peroxide levels were significantly more pronounced in males, due to lower levels of lipid peroxides among healthy males compared to female controls (males ME/CFS v. HC: CD19 B cells p=1.52*10-4, CD4 T cells p=5.92*10-4, CD8 T cells p=7.14*10-4, LC v. HC: CD19 B cells p=7.31*10-3, CD4 T cells p=9.39*10-3, CD8 T cells p=0.0114; females ME/CFS v. HC CD19 B cells p=0.0242, CD4 T cells p=0.0205, CD8 T cells p=0.00876; LC v. HC CD19 B cells p=0.622, CD4 T cells p=0.826, CD8 T cells p=0.974).

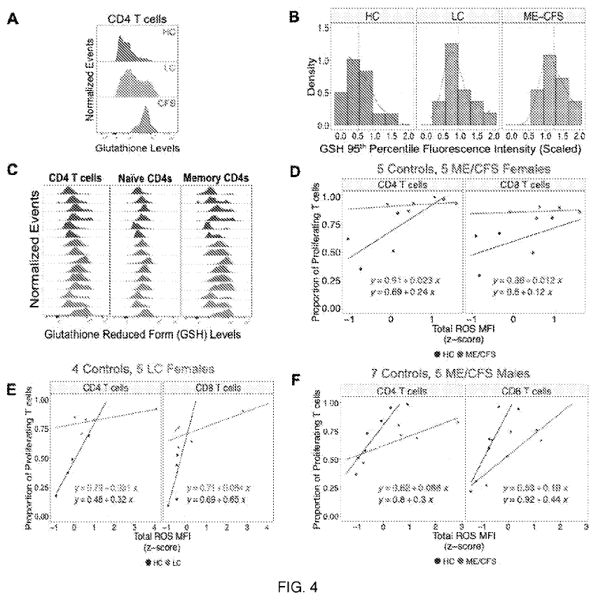

To protect membranes from lipid oxidative damage, the formation of lipid droplets can help restore redox homeostasis by sequestering ROS damaged lipids. Additionally, fatty acids derived from lipid droplets can be converted to acylcarnitines, the substrates for mitochondrial fatty acid oxidation. Based on our results in FIG. 3A, which also detected changes in acylcarnitine metabolism in ME/CFS donors, we compared lipid droplet levels using HCS LipidTox Green Neutral Lipid staining, which stains for neutral intracellular lipid droplets. As shown in FIG. 13G, we found lower lipid droplet levels in both LC and ME/CFS donors across all measured lymphocyte populations (ME/CFS: CD19 p=5.19*10⁻⁴, CD4 p=4.58*10⁻⁴, CD8 p=3.01*10⁻⁴; LC: CD19 p=0.140, CD4 p=0.0669, CD8 p=0.0759). Both ME/CFS and LC males and females presented with decreases in lipid droplet levels (FIG. 4G), although lipid droplet levels were higher in healthy females compared to healthy males (males ME/CFS v. HC: CD19 B cells p=1.52*10-4, CD4 T cells p=5.92*10-4, CD8 T cells p=7.14*10-4, LC v. HC: CD19 B cells p=7.31*10-3, CD4 T cells p=9.39*10-3, CD8 T cells p=0.0114; females ME/CFS v. HC CD19 B cells p=0.0242, CD4 T cells p=0.0205, CD8 T cells p=0.00876; LC v. HC CD19 B cells p=0.622, CD4 T cells p=0.826, CD8 T cells p=0.974). Furthermore, across both males and females, lipid droplet levels are inversely correlated with GSH levels across B and T lymphocytes. FIG. 3H shows the correlation across males and females (females: CD19 B cells R=−0.63, p=0.0072, CD4 T cells R=−0.67 p=0.003, CD8 T cells R=−0.54 p=0.025; males: CD19 B cells R=−0.9, p=2.5*10-5, CD4 T cells R=−0.75 p=0.0032, CD8 T cells R=−0.78, p=0.0015).

Our results correlating lipid droplet and glutathione levels link redox homeostasis with lipid composition and fatty acid oxidation, highlighting these metabolic consequences of oxidative stress. Furthermore, our findings highlight gender-specific redox differences that likely relate to distinct pathophysiology mechanisms among ME/CFS/LC males and females (FIG. 2A).

Elevated oxidative stress in memory CD4T and altered T cell proliferation responses to ROS in ME/CFS donors suggests a deficient adaptive immune response, upon stimulation. Beyond the metabolic effects, we also probed the functional consequences of excess oxidative stress in ME/CFS donor lymphocytes. It is known that elevated ROS are a critical, albeit transient component of both B and T cell lymphocyte activation. Specific to T cells, upon T cell activation, TCR signaling stimulates calcium influx into the mitochondria, which drives mitochondrial ROS production, activates NFAT signaling, and triggers IL2 cytokine production. Moreover, T cells without mitochondrial ROS are unable to proliferate upon antigen stimulation. Thus, the constitutive ROS elevation and alterations in mitochondrial calcium levels (FIG. 2B) that we see here in ME/CFS females suggests that that there will be differences in T cell proliferation compared to controls.

Next, re-examination of glutathione (GSH) data revealed significant variability in GSH distribution among CD4 T cells within each donor, especially among HC and LC patients (FIG. 4A). Comparing the extreme value distributions (95^{th} percentile fluorescence intensity) between HC, LC, and ME/CFS donor CD4 T cells, we observed that the HC CD4s decay most quickly (light-tailed distribution), whereas LC CD4s decay with a heavy-tailed distribution (FIG. 4B). In contrast, CFS donor CD4s have a shifted and bounded extreme value distribution. As comparisons of the extreme values alone rather than the medians or means identifies statistically significant elevation of GSH in LC and ME/CFS donors (HC v. LC CD4 p=0.00831; HC v. ME/CFS CD4 p=8.5*10⁻⁶), this result underscores that comparing the medians overlooks the critical heterogeneity that contributes to LC heavy-tail behavior. These results also suggest that a significantly larger proportion of T cells endure oxidative stress in ME/CFS donors, which is reflected in the shift in both comparisons of medians (FIG. 2D) and extreme value distributions (FIG. 4B). To assess which T cells may account for the differences in this tail phenomena, we compared naïve and memory CD4 T cell populations from 5 additional HC, 5 ME/CFS donors, and 4 LC subjects (FIG. 4C). Using CCR7 and CD45RO+ surface markers to delineate memory CD4 T cell populations (FIG. 10B), our analysis found that the long tails in CD4 GSH profiles were only reflected in memory and not naïve CD4 T cells (FIG. 4C). Moreover, ME/CFS and LC memory populations with a heavy tail have higher GSH values compared to their healthy counterparts. As antigen stimulation encourages the formation and recall of a T cell memory response, our results also suggested that ME/CFS donor T cells would likely respond distinctly to antigen stimulation.

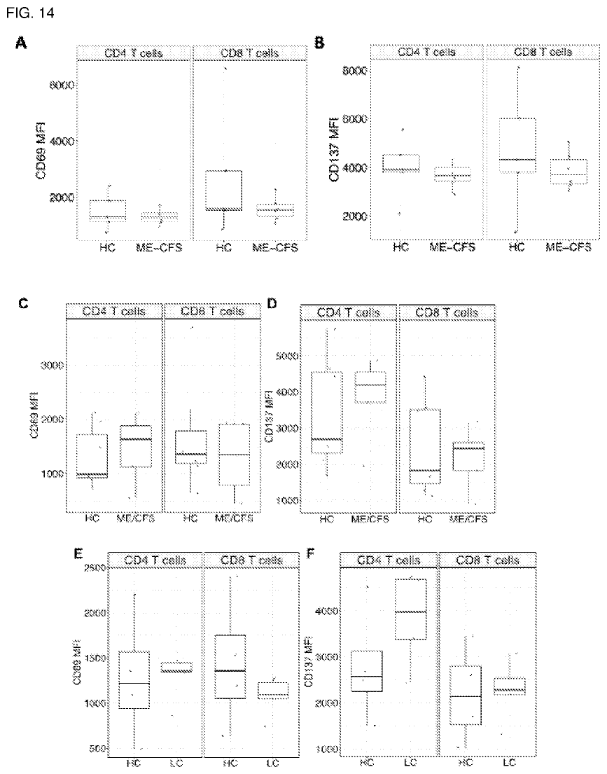

Therefore, we investigated the relation between oxidative stress in ME/CFS donor T cells and its proliferation, after stimulation. In light of our total ROS and mitochondrial calcium findings, we studied T cell proliferation in 5 HC and 5 ME/CFS female donor PBMCs. These PBMCs, which were labeled with CellTrace Violet proliferation dyes, were stimulated with anti-CD3/anti-CD28 antibodies and IL-2. The extent of proliferating T cells was measured 5 days post-stimulation, along with levels of oxidative stress and surface activation marker levels (CD69, CD137) in T cells (see methods for details). Separate experiments were conducted for ME/CFS females (FIG. 4D), LC females (FIG. 4E), and ME/CFS males (FIG. 4F). Upon stimulation, we observed no differences in CD69 and CD137 activation status for any ME/CFS and LC samples compared to HCs (FIG. 14). However, we noted sex-specific differences in the relationship between ROS and T cell proliferation, as described further below.

Consistent with the vital role of ROS in T cell activation, our analysis found among controls that the proportion of proliferating T cells linearly scales with oxidative stress (FIG. 4D-F). In contrast, the relation between ROS and T cell proliferation appears distinct in ME/CFS and LC subjects, with additional differences reflected between males and females. Compared to female controls (FIG. 4D showing 5 controls, 5 ME/CFS subjects), ME/CFS female CD4 and CD8 T cells show on average 26.5% and 28.6% higher proliferation respectively. Moreover, the regression lines in FIG. 4D show that CD4 and CD8 T cell proliferation among controls has 10× greater slope (blue line) with respect to ROS, compared to ME/CFS females (red). To check the generalizability of these findings, we evaluated in a separate experiment the relationship between ROS and T cell proliferation for 4 controls and 5 Long COVID female donors (FIG. 4E). Compared to controls, LC females on average show 39.7% and 42.8% higher CD4 and CD8 T cell proliferation, respectively. Consistent with the results in FIG. 4D, CD4 and CD8 T cell proliferation among controls has 10× greater slope (blue) with respect to ROS, compared to LC females. Together, these findings indicate that ME/CFS and LC T cells from female donors hyperproliferate, compared to controls, suggesting one potentially targetable source of patient fatigue.

In contrast, our analysis of 7 controls and 5 ME/CFS male donors found no differences in T cell proliferation on average (FIG. 4F). Comparing the slopes in the regression lines for FIG. 4F shows control male CD4 and CD8 T cell proliferation has 3.5 and 2.3× higher slope with respect to ROS, compared to ME/CFS males. While T cells from ME/CFS male donors do not hyperproliferate, their T cells exhibit greater insensitivity to ROS levels, compared to control counterparts.

These results suggest that the capacity for an individual ME/CFS T cell to proliferate is tuned differently or is insensitive to higher oxidative stress levels, pointing to a potential functional defect in ME/CFS T cell proliferation. As memory T cells account for the heterogeneity in the T cell glutathione profile and stimulation drives the formation of memory cells, our results imply deficient adaptive immune responses in ME/CFS donors. This finding agrees with an influenza vaccination study showing T cell hyperproliferation in the ME/CFS group versus controls, consistent with our result showing higher proportion of proliferating T cells upon in vitro stimulation. Additionally, characterization of stimulated CD4 T cells from LC donors12 found significantly higher levels of intracellular IL-2 levels, which are primarily produced from T cells through ROS-NFAT signaling. While neither of these cited studies probed for sex-specific differences in T cell proliferation, our findings highlight that the sex-specific pathways in redox dysregulation also shape the functional differences in male and female ME/CFS or LC adaptive immune responses, where female T cells hyperproliferate and male T cells have an insensitive response to ROS.

As T cell proliferation is associated with a 10-fold increase in energy usage due to elevated protein synthesis, these findings also point to a potential source of patient fatigue especially in ME/CFS female donors, where T cell activation drives rampant proliferation in CFS donors. Based on this hyperproliferation and the specific characterized pathways in FIGS. 2 and 3, we attempted to assess our findings in a clinical context and evaluated if ROS-modulating drugs could attenuate this hyperproliferation.

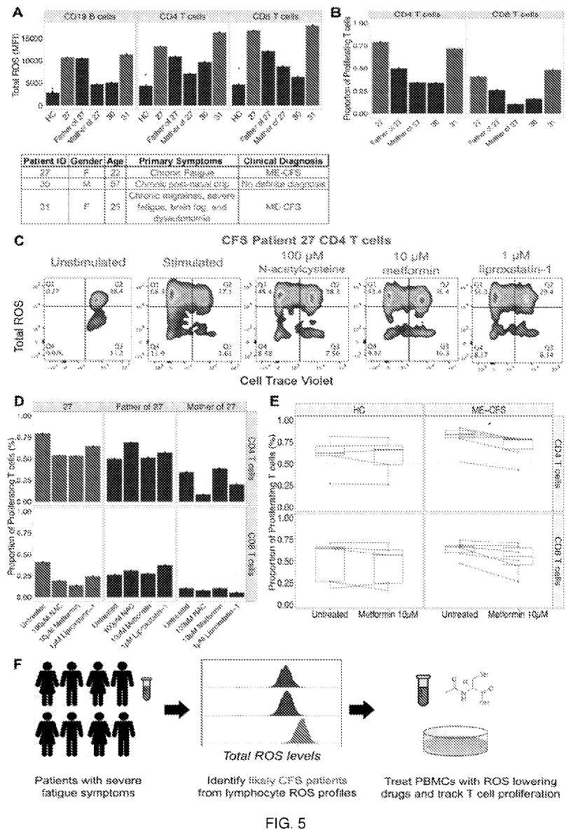

CFS donor T cell hyperproliferation can be attenuated with metformin. We evaluated total ROS levels in three patients (27, 30, 31) from the family population-omics profiling (fPOP) cohort, who presented with chronic symptoms (FIG. 5A, table). The clinical ME/CFS diagnosis, based on NAM criteria, was not available to us at the time of experimental characterization. Therefore, to contextualize changes in ROS levels, these donors were compared to 6 healthy controls (4 females, 2 males), along with samples from a ME/CFS presenting patient's (patient 27) mother and father.

Comparison of total ROS levels in flow cytometry enabled us to flag patient 27, the father of 27, and patient 31, based on elevation of total ROS levels (FIG. 5A). Overlaying our ROS data with patient symptoms showed that both patients 27 and 31 could be diagnosed as ME/CFS+, based on the NAM criteria. Additionally, patient 30, who exhibited chronic post-nasal drip but did not report any symptoms of fatigue, was not flagged by our assay, highlighting its sensitivity in distinguishing between patients with chronic symptoms (FIG. 5A). While our assay did not flag the mother of 27, our assay detected higher ROS levels in the father of 27 compared to healthy controls but still lower than patient 27.

As higher oxidative stress in T cells is associated with proliferation, we used CellTraceViolet staining to track the proliferation of fPOP donor T cells, upon anti-CD3/anti-CD28 and IL-2 stimulation. Expectedly, our findings showed a higher proportion of T cells from patients 27 and 31, who both exhibited elevated ROS and presented with ME/CFS symptoms, proliferated upon stimulation, compared to both parents of 27 and patient 30, who showed no fatigue symptoms and no elevation in ROS levels (FIG. 5B). These differences were observed across both CD4 and CD8 T cells, where ~1.8× and ~2.4× higher proportion of patient 27's CD4 and CD8 T cells respectively proliferated upon stimulation compared to her parents (CD4 T patient 27-79.3%, father of 27-50%, mother of 27-34.3%; CD8 T patient 27-41.0%, father of 27-26.0%, mother of 27-10.6%).

Based on the observation that ME/CFS donors have higher T cell ROS levels, associated with significantly higher proportion of proliferating T cells, we tested if ROS modulating drugs could lower the proportion of proliferating T cells. From our metabolic characterization (FIG. 2, 3), we tested three drugs, including N-acetylcysteine, metformin, and liproxstatin-1. N-acetylcysteine (NAC), an FDA approved drug, helps replenish glutathione levels, thereby acting as a critical reducing agent/anti-oxidant to combat oxidative stress. Metformin, also an FDA approved drug, inhibits mitochondrial complex I proteins, which subsequently induces SOD2 expression and reduces ROS formation. Our earlier assays identified SOD2 loss as a characteristic of LC and some ME/CFS donor lymphocytes (FIG. 2C) and double-blinded phase Ill studies on >1300 patients have shown metformin reduces Long COVID incidence by 41%, especially among female patients. Based on these results, metformin was also tested for its capacity to curtail T cell hyperproliferation in ME/CFS donors. Finally, as our lipid metabolic studies showed GPX4 changes and higher lipid peroxidation in ME/CFS and LC donors, we tested liproxstatin-1, a potent GPX4 modulator that can slow the accumulation of lipid peroxides.

Among the fPOP donors, we treated patient 27, 27's father, and 27's mother PBMCs at day 0 and compared the T cell proliferation after 5 days of stimulation between conditions. Comparing the stimulated conditions with treatment to the untreated for patient 27, our flow cytometry data suggested that all three drugs could modulate the proportion of high ROS proliferating T cells (FIG. 5C, Q1). Specifically, compared to 79.3% of proliferating CD4 T cells upon stimulation, NAC, metformin, and liproxstatin-1 treatment were able to lower this proportion to 54.3%, 53.2%, and 64.5% respectively. Moreover, we found that patient 27's T cell proliferation was uniquely responsive to these ROS-lowering drugs (FIG. 5D). In contrast, both patient 27's mother and father CD4 and CD8 proportion of proliferating T cells did not significantly change in response to any of these drugs (FIG. 5D).

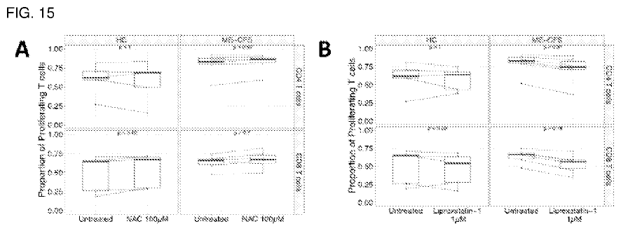

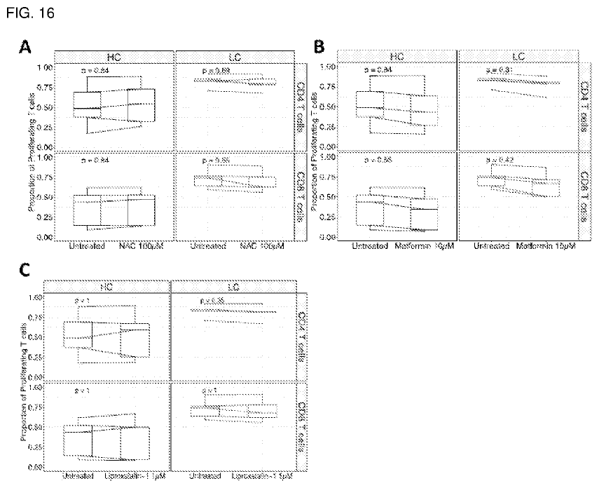

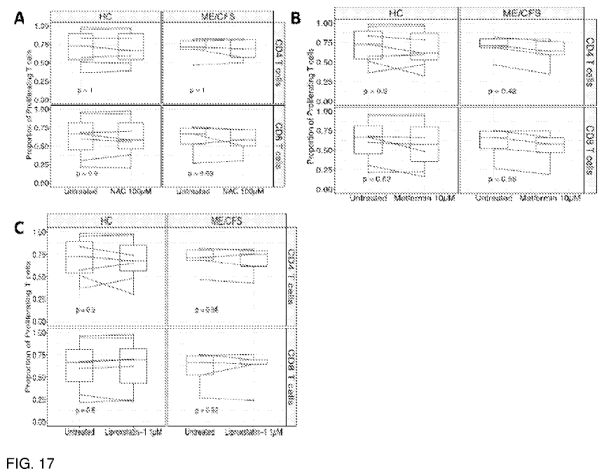

To test the generality of these findings, we tested these drugs on 5 HC and 6 ME/CFS donors, including those reported in FIG. 4D. While our assays found that NAC did not have any effect in reducing T cell hyperproliferation in ME/CFS donors (FIG. 15, ME/CFS CD4 p=0.82, CD8 p=0.7; HC CD4 p=1, CD8 p=0.42 metformin had a statistically significant effect in reducing CD4 T cell hyperproliferation in ME/CFS female donors (FIG. 5E, CD4 p=0.041, CD8 p=0.39). As NAC increases GSH levels, which are already significantly elevated in ME/CFS donors, our assays suggest that increasing glutathione levels is not sufficient to address the oxidative stress in ME/CFS donor lymphocytes. Also, our analysis on these 11 donors showed that metformin uniquely reduced the proliferation in ME/CFS donor T cells with no effect among the healthy controls (FIG. 5E, HC CD4 p=1, CD8 p=0.69). While liproxstatin-1 did not show a statistically significant effect in lowering T cell proliferation among female CFS or LC donors (FIGS. 15,16), the results again show that ME/CFS donor T cell proliferation is uniquely modulated by the drug and not healthy controls (FIG. 16, ME/CFS CD4 p=0.31, CD8 p=0.18; HC CD4 p=1, CD8 p=0.69). Metformin treatment had a smaller and non-statistically significant effect in LC female PBMCs (FIG. 16B, CD4 p=0.31, CD8 p=0.42), where treatment reduced on average CD4 and CD8 T cell proliferation in female LC donors by 5.2% and 7.6% respectively.

Consistent with the finding that male ME/CFS T cells did not hyperproliferate and displayed greater insensitivity to ROS, we did not observe any reduction in T cell proliferation upon treatment with NAC, metformin, or liproxstatin-1. Regardless, these findings demonstrate the possibility of a Precision Medicine approach for helping potentially diagnose and treat ME/CFS (FIG. 5F), especially among females. As shown in our study, tracking redox aberrations in lymphocytes can be used to identify individuals, who may present as ME/CFS based on clinical symptoms. Moreover, the selectivity of metformin and liproxstatin-1 in ME/CFS donors underscores that modulating oxidative stress is a tunable molecular link for controlling the extent of T cell proliferation in ME/CFS donors. It follows that tracking T cell proliferation in the context of ROS levels can be pinpoint which individuals may potentially benefit from ROS-lowering drugs and even identify novel drug candidates that mediate this link between ROS and T cell proliferation.

These findings demonstrate the use of a Precision Medicine approach for diagnosis and treatment of ME/CFS (FIG. 5F). As shown in our study, tracking redox aberrations in lymphocytes can be used to identify individuals, who may present as ME/CFS based on clinical symptoms. Moreover, the selectivity of metformin and liproxstatin-1 in ME/CFS donors underscores that modulating oxidative stress is a tunable molecular link for controlling the extent of T cell proliferation in ME/CFS donors. Tracking T cell proliferation in the context of ROS levels can be used to pinpoint which individuals will benefit from ROS-lowering drugs and identify novel drug candidates that mediate this link between ROS and T cell proliferation.

ME/CFS and Long Covid share many clinical features and are increasingly diagnosed together, but there have been no quantitative diagnostic tests and clues as to a shared basis have been lacking. Previous studies have hypothesized oxidative stress as a common biochemical signature and even shown aberrations in circulating serum redox proteins (e.g., myeloperoxidase) among LC patients. Unique to our work, the analysis of intracellular pathways shows that both diseases share signatures of elevated oxidative stress, specifically among lymphocytes, compared to healthy controls that would impact mitochondrial function. Additional intracellular metabolic characterization using a variety of approaches shows that these changes are reflected in multiple oxidative stress pathways, including alterations in glutathione, superoxide dismutase, lipid oxidative damage, etc. Our work is the first to pinpoint a targetable cellular mechanism, where these metabolic changes clearly affect lymphocyte activation and immune responses generally, explaining the prevalence of fatigue and other symptoms in these two syndromes.

Our findings highlight sex-specific redox signatures in ME/CFS patients, such as elevated total ROS levels in women versus men, suggesting that the pathophysiology for ME/CFS and LC are distinct between genders. Published studies in LC and ME/CFS have shown redox abnormalities in circulating components, including serum proteins and erythrocytes. Moreover, estrogen also shapes the activation of CD4 T cells, which express the estrogen receptor (ERα). Comparison of in-vitro stimulation of T cells between males and females shows higher expression of anti-viral genes in females (IFNG, RIGI, OAS1) and higher production of IFN□ upon stimulation62. Additionally, both our study and a recent deep phenotyping study on ME/CFS patients show differences in fatty acid oxidation pathways between ME/CFS males and females, with the results here uniquely highlighting larger changes in lipid peroxidation. As androgens modulate peroxisome proliferator-activated receptor (PPAR) a levels in CD4 T cells, where PPARα critically regulates the production of fatty acid oxidation enzymes, sex differences in PPARα CD4 T cells may explain this difference in lipid metabolism.

These metabolic differences may impair the adaptive immune response in ME/CFS and LC individuals. Through studying extreme value distributions of the glutathione profile, our findings identify memory CD4 T cells as critical contributors to the heterogeneity in bulk CD4 population profiles. These findings, which show LC CD4 T cells oxidative profiles exhibit a long-tail phenomena, also caution against solely comparing medians/means between donors, which do not capture this heterogeneity. By comparing T cell proliferation upon antigen stimulation, we find oxidative stress linearly scales with the proportion of proliferating T cells, meaning a higher fraction of female CFS donor T cells proliferate upon stimulation. In addition, our results showing altered sensitivity of T cell proliferation to oxidative stress levels among CFS donors (compared to HCs) carries additional implications. With the higher energy demands needed for T cell proliferation, these differences suggest the availability of energy as a significant and unique contributor to fatigue in CFS donors. We suggest that this lymphocyte dysfunction, driven by oxidative and mitochondrial damage, acts as an “energy sink”, much as an active infection does, draining the body of available energy and giving rise to debilitating fatigue and other sequelae. Moreover, it suggests that at least in females, ROS levels may serve as a tunable link for adjusting T cell proliferation, with CFS donor T cells showing insensitivity to higher ROS levels.

As demonstrated in the fPOP study, our work demonstrates a Precision Medicine methodology to identify specific pre-existing FDA approved and novel candidates that can adjust ROS levels and subsequently curtail T cell hyperproliferation in CFS donors. Our results offer a mechanism of action in LC patients and more broadly show that the link between oxidative stress and aberrant T cell proliferation can be exploited to identify novel drug candidates.

Although our findings were focused on identifying a conserved signature between ME/CFS and LC patients, based on commonly presented symptoms, our analysis suggests several important differences. For example, while both ME/CFS and LC donors show elevated ROS levels in T cells, the specific aberrant anti-oxidant pathways appear to differ slightly, where LC patients overall show lower mitochondrial superoxide dismutase and calcium levels compared to ME/CFS donors. Additionally, our comparison of the extreme value distributions found that LC T cells show a heavy-tailed oxidative stress profile and CFS donor T cells show a shifted but bounded extreme value distribution. As one difference between these groups in our study corresponds to the duration of symptoms, LC donors may capture an intermediate state between HC and ME/CFS donors. One interpretation of this concept is that continuous exposure to high oxidative stress in lymphocytes may cultivate tolerance to ROS, as a possible protective mechanism for some patients. This tolerance may consequently lead to insensitivity to ROS levels, upon stimulation, as evidenced by our findings in ME/CFS T cells.

These results also raise the question as to how these oxidative stress pathways are triggered and maintained. Since Long Covid is clearly triggered by an infection, the initial elevation of ROS levels necessary for lymphocyte activation may be overly prolonged in some individuals, causing damage to mitochondrial membrane physiology. This ROS elevation may then return to baseline (or get cleared more rapidly) in males but not females, but in both sexes with the LC syndrome the damage is long lasting. In ME/CFS it has long been thought that there was a causative infection, although a specific pathogen has not been identified, despite intensive efforts. Nevertheless, it has been noted that other infections have produced LC-like symptoms in particular individuals, so the pathologies we document here might be a feature of other infections as well. It may be that individuals with sub clinical immune deficiencies suffer from a more prolonged infection and accompanying ROS elevation that produces lasting mitochondrial damage.

These results also raise the question as to how these oxidative stress pathways are triggered and maintained. Here we can only speculate that since Long Covid is clearly triggered by an infection, the initial elevation of ROS levels necessary for lymphocyte activation is overly prolonged in some individuals, perhaps caused by an impaired ability to clear the virus, causing damage to mitochondrial membrane physiology. This ROS elevation may then return to baseline (or get cleared more rapidly) in males but not females, but in both sexes with the LC syndrome the damage is long lasting. In ME/CFS it has long been thought that there was a causative infection, although a specific pathogen has not been identified, despite intensive efforts. Nevertheless, it has been noted that other infections have produced LC-like symptoms in particular individuals, so the pathologies we document here might be a feature of other infections as well. It may be that individuals with sub clinical immune deficiencies suffer from a more prolonged infection and accompanying ROS elevation that produces lasting mitochondrial damage.

### Methods

Patient Description: For the current study, we selected three cohorts (FIG. 1A). Healthy control (HC) samples were obtained from the Stanford Blood Bank under IRB-40146. HC participants must be 17 years of age or older and complete a medical history questionnaire that includes recent symptoms related to COVID-19 or a positive test two weeks before the blood donation and test negative for hepatitis B and C, HIV, HTLV, syphilis, and West Nile Virus.

ME/CFS patients were eligible for inclusion in the study under IRB-40146 if they had been diagnosed using the National Academy of Medicine and Canadian Consensus Criteria (CCC). Severity was assessed using Bell's disability scale. Informed consent was obtained through REDCap, and study data were collected and managed using REDCap electronic data capture tools hosted at the ME/CFS Collaborative Research Center at Stanford University.

Long COVID-19 participants were patients from the Stanford Post-acute COVID syndrome (PACS) clinic. These patients met the CDC case definition of Long COVID, must be 18 years of age or older, must have new symptoms after SARS-CoV-2 infection and evidence of positive infection (positive PCR, antigen test, or antibodies before SARS-CoV-2 vaccination). Blood collection, demographic, and clinical data were collected from this study cohort under IRB approval protocol (IRB-64344) and this information was blinded to the bench researchers. Based on the National Academy of Medicine criteria, 9/15 (60%) met the diagnostic criteria for ME/CFS.

Clinical Cohorts Demographics

| | Healthy | Chronic Fatigue | Long |
|---|---|---|---|
| Clinical | Controls | Syndrome | COVID |
| Cohorts | (HC) | (ME/CFS) | (LC) |
| Number of | 16 | 15 | 15 |
| Participants | | | |
| Mean age | 53 | 52 | 47 |
| % female | 46.6% | 76.9% | 53.3 |
| % ME/CFS | N/A | 15/15 (100%) ** | 9/15 (60%) * |

* IOM criteria,
** IOM and CC criteria,
N/A Non applicable

PBMC Preparation: Human primary peripheral blood mononuclear cells (PBMCs) were isolated from the whole blood of both healthy donors and ME-CFS patients using SepMate™ 50 mL Tubes, following the protocol specifications. After isolation, cells were frozen in FBS+10% DMSO and stored in liquid nitrogen tanks.

PBMCs were thawed and washed in pre-warmed RPMI media with benzonase. RPMI media included RPMI-1640 media supplemented with 10% dialyzed FBS, 1% glutamax, and 1% penicillin/streptomycin. After spinning down cells at 400 g for 5 minutes, cells were counted and viability was evaluated by Trypan blue staining. Cells were passed through a 70 μm cell strainer, spun down at 400 g for 5 minutes, and re-suspended at 1-2 million cells/well in RPMI media. After resting PBMCs overnight for 12-18 hrs at 37 C, cells were stained the following morning with metabolic dyes.

Staining: To stain for mitochondrial parameters, cells were stained with BioTracker ATP-Red Live Cell Dye (Sigma Aldrich), MitoTracker Deep Red FM (ThermoFisher), MitoSpy Green FM (BioLegend). To stain for ROS levels, cells were stained with DCFDA cellular ROS assay kit (abcam), MitoSOX Red Mitochondrial Superoxide detector (ThermoFisher). To stain for glutathione clearance and oxidative damage, cells were stained with ThiolTracker Violet (ThermoFisher), HCS LipidTOX Green Neutral Lipid Stain (ThermoFisher) for lipid droplet levels, and Image-iT™ Lipid Peroxidation Kit, ratiometric lipid peroxidation sensor (ThermoFisher). To capture viability, cells were stained with LIVE/DEAD Fixable Far Red Dead Cell Stain (ThermoFisher).

Cells were then collected, spun down 2× at 400 g for 5 minutes, stained with live-dead dye and Fc receptor (FcR) block in FACS buffer (PBS, 10% dialyzed FBS, 1 mM EDTA). After 10 minutes, cells were subsequently stained with surface antibodies for 30 minutes. Due to the variety of metabolic dyes used, we also set-up multiple panels accordingly. We list all antibodies used across any panel. Antibodies included anti-CD3 (BUV805-UCHT-1, BV605-OKT3), anti-CD8 (BUV395, RPA-T8; BUV737, RPA-T8), anti-CD4 (BV650, RPA-T4, AlexaFluor700, RPA-T4), anti-CD19 (PerCP/Cy5.5, HIB19). For single colored controls, combination of compensation beads and stained cells were used for surface antibodies and metabolic dyes, respectively. Naïve and memory CD4 T cells were stained using anti-CCR7 (PerCP/Cy5-5, Clone G043H7; BV421, Clone G043H7) and anti-CD45RO (PE/Cy7, UCHL1, BioLegend) antibodies. Activated T cells were stained in proliferation assays using anti-CD69 (BUV395; Clone FN50) and anti-CD137 (BV750, Clone-4B4-1). Flow cytometric and fluorescence activated cell sorting analysis was performed on an BD Aria Fusion sorter and BD FACS Symphony A5 instruments. Flow cytometry data was analyzed using FlowJo software (BD). Staining levels were compared using median and maximum fluorescence intensity (MFI).

Intracellular Staining for SOD2/Ki-67: After staining PBMCs with surface markers and live-dead dyes, cells were fixed and permeabilized, using BD Cytofix/Cytoperm kit. Subsequently, cells were stained with antibodies to intracellular proteins (SOD2 (FITC, Clone: 3A6C2, ThermoFisher); Ki-67 (PEDazzle594, Clone: Ki-67, BioLegend) for 45 minutes at 4 C.

Immunofluorescence Staining Microscopy. Upon CD3 (Alexa594, UCHT1, BioLegend) and GPX4 staining (ThermoFisher, Catalog ID: PA5-102521), stained cells were transferred to microscope slides, using cytospin preparation. Images were acquired using a Leica DMi8 Thunder Imager equipped with a Leica DFC9000 GT Camera. Z-stack images were rendered and segmented in three-dimensional space using AIVIA Pixel Classifier and Recipe Analysis for subcellular signal quantification.

Proliferation Assays: PBMCs were labeled with proliferation dye according to manufacturer's instructions using CellTrace Violet (ThermoFisher). After resting labeled PBMCs for an hour, cells were stimulated with anti-CD3/anti-CD28 antibodies (StemCell Technologies) with IL-2 50 IU/mL. The extent and proportion of proliferating T cells were compared 5 days after stimulation.

Mass Spectrometry: First, 200,000 CD3 T cells were sorted, pooled across several healthy control and ME-CFS donors. Metabolites were immediately extracted from cells using 80% methanol and 20% water mixture with mass spectrometry grade internal standards (Ref. 23, main text). The cells are repeatedly vortexed and sonicated, upon which the proteins are precipitated, and extracted metabolites are dried under nitrogen stream and stored in −80° C. until ready to run. The hydrophilic interaction (HILIC)-MS analysis was conducted using a Thermo UltiMate 3000 UHPLC coupled to a Thermo Q Exactive HF mass spectrometer. HILIC experiments were performed using a ZIC-HILIC column (2.1×100 mm, 3.5 μm, 200 Å; Merck Millipore) with mobile phase solvents consisting of 10 mM ammonium acetate in 50/50 acetonitrile/water (A) and 10 mM ammonium acetate in 95/5 acetonitrile/water (B). Peaks were assigned by matching m/z and retention time with external databases and pre-purchased analytical standards. The fragmentation profile for all peaks were compared with several databases, including HMDB, METLIN, Lipid MAPS, and MassBank to assign the peak identities.

Turium: This is a system chemistry tool developed for complex chemical analysis. After the identification of analytes, mass spectrometry data was processed for Turium analysis, where metabolic networks were constructed of healthy controls and ME-CFS patients. From 45,507 unique detected analytes, 747 were identified based on comparisons with known analytical standards (Ref. 23, main text), and 327 different metabolic pathways were mapped. The resulting specific metabolic reactions were checked with KEGG Pathway Database. The difference between the metabolic pathways, those in ME-CFS donors but not in healthy controls, was computed and visualized using network package in R v3.6.3.

### Example 2

Connections to Autoimmunity, where Patients Also Present with Debilitating Fatigue

A major shared symptom among most autoimmune patients is fatigue. According to one estimate from the American Autoimmune and Related Disorders Association survey of 7,838 autoimmune patients, 98% of surveyed patients experience fatigue, with ~60% reporting “debilitating fatigue”. In this regard, the clinical presentation of chronic autoimmune diseases overlaps with chronic fatigue syndrome, a complex multisystemic condition, where patients report disabling fatigue and post-exertional malaise. There are no treatments for fatigue, although its link with energy metabolism and inflammation are strongly suspected. As the immune system accounts for 15-20% of the daily energy expenditure, we hypothesize that patient fatigue corresponds to metabolic derangements that are even reflected in the immune response. Specifically, our core hypothesis among autoimmune patients is that metabolic derangements in lymphocytes, especially oxidative stress and redox imbalance, can drive patient fatigue, due to the energy required to sustain a chronic inflammatory stage.

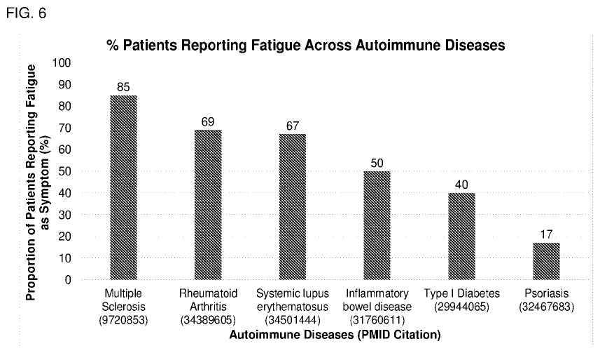

As fatigue is shared between chronic fatigue syndrome (ME-CFS), Long COVID (LC) patients, who report disabling and persistent fatigue for months after testing COVID negative post-infection, and over two-thirds of SLE patients (FIG. 6), we initially asked whether there might be shared metabolic deficiencies between ME-CFS (n=5), Long COVID (n=5), SLE (n=3) donors, versus healthy controls (HC) (n=5). Peripheral blood mononuclear cells (PBMCs) were obtained from subjects and thawed overnight. Samples were subsequently stained with surface antibodies to define immune cell populations and several metabolic probes, including ones to measure cellular metabolic and mitochondrial properties. The stained samples were then characterized using flow cytometry, which enabled comparisons of cell populations and their metabolic states.

Using fluorogenic probes to compare mitochondrial ATP levels revealed resting SLE lymphocytes have 1.5× lower ATP levels, compared to healthy controls. As both Long COVID and ME-CFS donors also show 2.8× and 1.6× lower mitochondrial ATP levels compared to the same set of healthy controls, this data highlights mitochondrial functional deficits are common among these patients experiencing persistent fatigue.

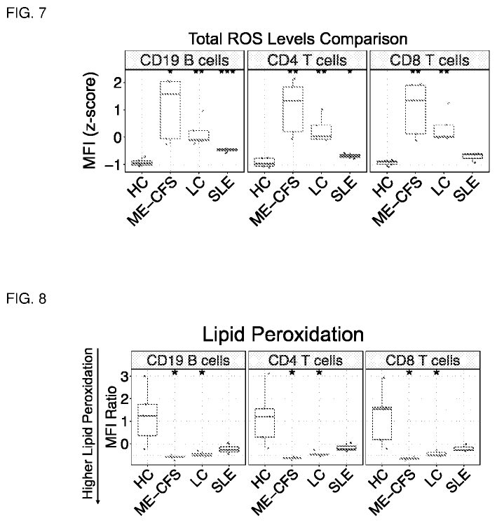

Using another fluorogenic probe dichlorodihydrofluorescein diacetate (DCFDA), oxidized primarily by hydrogen peroxide, FIG. 7 compares the total ROS levels among lymphocytes between the different patient groups by condition. Notably, our results show that SLE donor lymphocytes have higher total ROS (1.6× higher on average) compared to healthy controls (CD19 B cells p=0.001, CD4 T cells p=0.044, CD8 T cells p=0.158) (FIG. 7) but are lower compared to ME-CFS and LC donor-derived lymphocytes.

To probe the metabolic consequences of higher oxidative stress, we also tested for signs of oxidative damage, namely irreversible damage to lipids based on the oxidation of fatty acids due to the presence of excess reactive oxygen species. Flow cytometric experiments using a ratiometric fluorescence sensor to detect lipid oxidative damage suggests that SLE donor lymphocytes exhibit >2× higher lipid peroxide levels, compared to healthy controls (FIG. 8, lower ratio implies higher lipid peroxidation; SLE CD19 B cells p=0.058, CD4 T cells p=0.074, CD8 T cells p=0.064).

Our data show that oxidative damage appears to be a common signature among ME-CFS, LC, and SLE lymphocytes, highlighting that patients presenting with symptoms of fatigue share a metabolic defect in lymphocytes.

### REFERENCES

Davis H E, McCorkell L, Vogel J M, Topol E J. Long COVID: major findings, mechanisms and recommendations. Nat Rev Microbiol. 2023 Jan. 13. PMID: 36639608.
Nasserie T, Hittle M, Goodman S N. Assessment of the Frequency and Variety of Persistent Symptoms Among Patients With COVID-19: A Systematic Review. JAMA Network Open. 2021 May 3; 4(5):e2111417. PMID: 34037731; PMCID: PMC8155823.
Xie Y, Xu E, Bowe B, Al-Aly Z. Long-term cardiovascular outcomes of COVID-19. Nat Med. 2022 March; 28(3):583-590. PMID: 35132265; PMCID: PMC8938267.
Fernández-Castañeda et al. Mild respiratory COVID can cause multi-lineage neural cell and myelin dysregulation. Cell. 2022 Jul. 7; 185(14):2452-2468.e16. doi: 10.1016/j.cell.2022.06.008. Epub 2022 Jun. 13. PMID: 35768006; PMCID: PMC9189143.
Aiyegbusi O L, et al. Symptoms, complications and management of long: 10.1177/01410768211032850. Epub 2021 Jul. 15. PMID: 34265229; PMCID: PMC8450986.
Choutka J, Jansari V, Hornig M, Iwasaki A. Unexplained post-acute infection syndromes. Nat Med. 2022 May; 28(5):911-923. doi: 10.1038/s41591-022-01810-6. Epub 2022 May 18. Erratum in: Nat Med. 2022 August; 28(8):1723. PMID: 35585196.
Institute of Medicine (2015). Beyond Myalgic Encephalomyelitis/Chronic Fatigue Syndrome: Redefining an Illness (The National Academies Press, Washington, DC).
Chang C J, et al. A Comprehensive Examination of Severely III ME/CFS Patients. Healthcare (Basel). 2021 Sep. 29; 9(10):1290. PMID: 34682970; PMCID: PMC8535418.
Kedor C, et al. A prospective observational study of post-COVID-19 chronic fatigue syndrome following the first pandemic wave in Germany and biomarkers associated with symptom severity. Nat Commun. 2022 Aug. 30; 13(1):5104. doi: 10.1038/s41467-022-32507-6.
Montoya J G et al. Cytokine signature associated with disease severity in chronic fatigue syndrome patients. Proc Natl Acad Sci USA. 2017 Aug. 22; 114(34):E7150-E7158. doi: 10.1073/pnas.1710519114. Epub 2017 Jul. 31. PMID: 28760971; PMCID: PMC5576836.
Talla A et al. Persistent serum protein signatures define an inflammatory subcategory of long COVID. Nat Commun. 2023 Jun. 9; 14(1):3417. doi: 10.1038/s41467-023-38682-4. PMID: 37296110; PMCID: PMC10252177.
Klein J, et al. Distinguishing features of Long COVID identified through immune profiling. Nature. 2023 Sep. 25. doi: 10.1038/s41586-023-06651-y. PMID: 37748514.
Altmann D M, Whettlock E M, Liu S, Arachchilage D J, Boyton R J. The immunology of long COVID. Nat Rev Immunol. 2023 October; 23(10):618-634. doi: 10.1038/s41577-023-00904-7. Epub 2023 Jul. 11. Erratum in: Nat Rev Immunol. 2023 Sep. 18; PMID: 37433988.
Wong A C, et al. Serotonin reduction in post-acute sequelae of viral infection. Cell. 2023 Oct. 9: S0092-8674(23)01034-6.
Xiong R, Gunter C, Fleming E, Vernon S D, Bateman L, Unutmaz D, Oh J. Multi-'omics of gut microbiome-host interactions in short- and long-term myalgic encephalomyelitis/chronic fatigue syndrome patients. Cell Host Microbe. 2023 Feb. 8; 31(2):273-287.e5 PMID: 36758521; PMCID: PMC10353054.
Guo C, et al. Deficient butyrate-producing capacity in the gut microbiome is associated with bacterial network disturbances and fatigue symptoms in ME/CFS. Cell Host Microbe. 2023 Feb. 8; 31(2):288-304.e8. PMID: 36758522; PMCID: PMC10183837.
Naviaux R K, Naviaux J C, Li K, Bright A T, Alaynick W A, Wang L, Baxter A, Nathan N, Anderson W, Gordon E. Metabolic features of chronic fatigue syndrome. Proc Natl Acad Sci USA. 2016 Sep. 13; 113(37):E5472-80. doi: 10.1073/pnas. 1607571113. Epub 2016 Aug. 29. Erratum in: Proc Natl Acad Sci USA. 2017 May 2; 114(18):E3749. PMID: 27573827; PMCID: PMC5027464.
Straub R H. The brain and immune system prompt energy shortage in chronic inflammation and ageing. Nat Rev Rheumatol. 2017 December; 13(12):743-751. doi: 10.1038/nrrheum.2017.172. Epub 2017 Oct. 12. PMID: 29021568.
Straub R H, Cutolo M, Buttgereit F, Pongratz G. Energy regulation and neuroendocrine-immune control in chronic inflammatory diseases. J Intern Med. 2010 June; 267(6):543-60. doi: 10.1111/j.1365-2796.2010.02218.x. Epub 2010 Jan. 28. PMID: 20210843.
Nagy-Szakal D, et al. Insights into myalgic encephalomyelitis/chronic fatigue syndrome phenotypes through comprehensive metabolomics. Sci Rep. 2018 Jul. 3; 8(1):10056. doi: 10.1038/s41598-018-28477-9. PMID: 29968805; PMCID: PMC6030047.
Buck M D, et al. Mitochondrial Dynamics Controls T Cell Fate through Metabolic Programming. Cell. 2016 Jun. 30; 166(1):63-76. doi: 10.1016/j.cell.2016.05.035. Epub 2016 Jun. 9. PMID: 27293185; PMCID: PMC4974356.
Naik E, Dixit V M. Mitochondrial reactive oxygen species drive proinflammatory cytokine production. J Exp Med. 2011 Mar. 14; 208(3):417-20. doi: 10.1084/jem.20110367. Epub 2011 Feb. 28. PMID: 21357740; PMCID: PMC3058577.
Guarnieri J W et al. Core mitochondrial genes are down-regulated during SARS-CoV-2 infection of rodent and human hosts. Sci Transl Med. 2023 Aug. 9; 15(708):eabq1533. doi: 10.1126/scitranslmed.abq1533. Epub 2023 Aug. 9. PMID: 37556555.
Jahanbani F, et al. Phenotypic characteristics of peripheral immune cells of Myalgic encephalomyelitis/chronic fatigue syndrome via transmission electron microscopy: A pilot study. PLoS One. 2022 Aug. 9; 17(8):e0272703. doi: 10.1371/journal.pone.0272703. PMID: 35943990; PMCID: PMC9362953.
Díaz-Resendiz K J G, et al. Loss of mitochondrial membrane potential (Δψₘ) in leucocytes as post-COVID-19 sequelae. J Leukoc Biol. 2022 July; 112(1):23-29. doi: 10.1002/JLB.3MA0322-279RRR. Epub 2022 Mar. 31. PMID: 35355308; PMCID: PMC9088601.
Paul B D, Lemle M D, Komaroff A L, Snyder S H. Redox imbalance links COVID-19 and myalgic encephalomyelitis/chronic fatigue syndrome. Proc Natl Acad Sci USA. 2021 Aug. 24; 118(34):e2024358118. doi: 10.1073/pnas.2024358118. PMID: 34400495; PMCID: PMC8403932.
Klok F A, Boon G J A M, Barco S, Endres M, Geelhoed J J M, Knauss S, Rezek S A, Spruit M A, Vehreschild J, Siegerink B. The Post-COVID-19 Functional Status scale: a tool to measure functional status over time after COVID-19. Eur Respir J. 2020 Jul. 2; 56(1):2001494. doi: 10.1183/13993003.01494-2020. PMID: 32398306; PMCID: PMC7236834.
Mittal M, Siddiqui M R, Tran K, Reddy S P, Malik A B. Reactive oxygen species in inflammation and tissue injury. Antioxid Redox Signal. 2014 Mar. 1; 20(7):1126-67. doi: 10.1089/ars.2012.5149. Epub 2013 Oct. 22. PMID: 23991888; PMCID: PMC3929010.
David S, Bell M. The Doctor's Guide to Chronic Fatigue Syndrome, pp. 122-4.
Unger E R, Lin J S, Brimmer D J, et al. CDC Grand Rounds: Chronic Fatigue Syndrome—Advancing Research and Clinical Education. MMWR Morb Mortal Wkly Rep 2016; 65:1434-1438. DOI: http://dx.doi.org/10.15585/mmwr.mm655051a4.
Bai F, et al. Female gender is associated with long COVID syndrome: a prospective cohort study. Clin Microbiol Infect. 2022 April; 28(4):611.e9-611.e16. doi: 10.1016/j.cmi.2021.11.002. Epub 2021 Nov. 9. PMID: 34763058; PMCID: PMC8575536.
Sylvester S V et al. Curr Med Res Opin. 2022 August; 38(8):1391-1399. doi: 10.1080/03007995.2022.2081454. PMID: 35726132.
Debski M et al. PLOS Glob Public Health. 2022 Nov. 30; 2(11):e0001188. doi: 10.1371/journal.pgph.0001188. PMID: 36962824; PMCID: PMC10022108.
Görlach A, Bertram K, Hudecova S, Krizanova O. Calcium and ROS: A mutual interplay. Redox Biol. 2015 December; 6:260-271. doi: 10.1016/j.redox.2015.08.010. Epub 2015 Aug. 11. PMID: 26296072; PMCID: PMC4556774.
Fukai T, Ushio-Fukai M. Superoxide dismutases: role in redox signaling, vascular function, and diseases. Antioxid Redox Signal. 2011 Sep. 15; 15(6):1583-606. doi: 10.1089/ars.2011.3999. Epub 2011 Jun. 6. PMID: 21473702; PMCID: PMC3151424.
Polonikov A. Endogenous Deficiency of Glutathione as the Most Likely Cause of Serious Manifestations and Death in COVID-19 Patients. ACS Infect Dis. 2020 Jul. 10; 6(7):1558-1562. doi: 10.1021/acsinfecdis.0c00288. Epub 2020 May 28. PMID: 32463221; PMCID: PMC7263077.
Ubezio P, Civoli F. Flow cytometric detection of hydrogen peroxide production induced by doxorubicin in cancer cells. Free Radic Biol Med. 1994 April; 16(4):509-16. doi: 10.1016/0891-5849 (94) 90129-5. PMID: 8005536.
Sharifi-Rad M, et al. J. Lifestyle, Oxidative Stress, and Antioxidants: Back and Forth in the Pathophysiology of Chronic Diseases. Front Physiol. 2020 Jul. 2; 11:694. doi: 10.3389/fphys.2020.00694. PMID: 32714204; PMCID: PMC7347016.
Contrepois K, Jiang L, Snyder M. Optimized Analytical Procedures for the Untargeted Metabolomic Profiling of Human Urine and Plasma by Combining Hydrophilic Interaction (HILIC) and Reverse-Phase Liquid Chromatography (RPLC)-Mass Spectrometry. Mol Cell Proteomics. 2015 June; 14(6):1684-95. doi: 10.1074/mcp.M114.046508. Epub 2015 Mar. 18. PMID: 25787789; PMCID: PMC4458729.
V. Shankar, S. Shankar, A Novel Computational Artificial Intelligence Framework for Complex Physical, Chemical and Biological Networks, APS March Meeting, (2021).
Bouquet J, et al. Whole blood human transcriptome and virome analysis of ME/CFS patients experiencing post-exertional malaise following cardiopulmonary exercise testing. PLoS One. 2019 Mar. 21; 14(3):e0212193. doi: 10.1371/journal.pone.0212193. PMID: 30897114.
Catalá A. Lipid peroxidation of membrane phospholipids generates hydroxy-alkenals and oxidized phospholipids active in physiological and/or pathological conditions. Chem Phys Lipids. 2009 January; 157(1):1-11. doi: 10.1016/j.chemphyslip.2008.09.004. Epub 2008 Oct. 14. PMID: 18977338.
Jarc E, Petan T. Lipid Droplets and the Management of Cellular Stress. Yale J Biol Med. 2019 Sep. 20; 92(3):435-452. PMID: 31543707; PMCID: PMC6747940.
Franchina D G, Dostert C, Brenner D. Reactive Oxygen Species: Involvement in T Cell Signaling and Metabolism. Trends Immunol. 2018 June; 39(6):489-502. doi: 10.1016/j.it.2018.01.005. Epub 2018 Feb. 13. PMID: 29452982.
Yarosz E L, Chang C H. The Role of Reactive Oxygen Species in Regulating T Cell-mediated Immunity and Disease. Immune Netw. 2018 Feb. 22; 18(1):e14. doi: 10.4110/in.2018.18.e14. PMID: 29503744; PMCID: PMC5833121.
Sena L A, et al. Mitochondria are required for antigen-specific T cell activation through reactive oxygen species signaling. Immunity. 2013 Feb. 21; 38(2):225-36. doi: 10.1016/j.immuni.2012.10.020. Epub 2013 Feb. 15. PMID: 23415911; PMCID: PMC3582741.
A. Hansen, The three extreme value distributions: An introductory review, Front. Phys. 8, 533 (2020).
Marchingo J M, Cantrell D A. Protein synthesis, degradation, and energy metabolism in T cell immunity. Cell Mol Immunol. 2022 March; 19(3):303-315. doi: 10.1038/s41423-021-00792-8. Epub 2022 Jan. 4. PMID: 34983947; PMCID: PMC8891282.
Wolf T, et al. Dynamics in protein translation sustaining T cell preparedness. Nat Immunol. 2020 August; 21(8):927-937. doi: 10.1038/s41590-020-0714-5. Epub 2020 Jul. 6. PMID: 32632289; PMCID: PMC7610365.
Gu, X., et al. (2023). Probing long COVID through a proteomic lens: A comprehensive two-year longitudinal cohort study of hospitalised survivors. eBioMedicine, 98, 104851. https://doi.org/10.1016/j.ebiom.2023.104851
Prinsen H, et al. Humoral and cellular immune responses after influenza vaccination in patients with chronic fatigue syndrome. BMC Immunol. 2012 Dec. 17; 13:71. doi: 10.1186/1471-2172-13-71. PMID: 23244635; PMCID: PMC3534525.
Chen R, et al. Personal omics profiling reveals dynamic molecular and medical phenotypes. Cell. 2012 Mar. 16; 148(6):1293-307. doi: 10.1016/j.cell.2012.02.009. PMID: 22424236; PMCID: PMC3341616.
Geng Y, Hernández Villanueva A, Oun A, Buist-Homan M, Blokzijl H, Faber K N, Dolga A, Moshage H. Protective effect of metformin against palmitate-induced hepatic cell death. Biochim Biophys Acta Mol Basis Dis. 2020 Mar. 1; 1866(3):165621. doi: 10.1016/j.bbadis.2019.165621. Epub 2019 Nov. 29. PMID: 31786336.
Bramante C T, et al. Outpatient treatment of COVID-19 and incidence of post-COVID-19 condition over 10 months (COVID-OUT): a multicentre, randomised, quadruple-blind, parallel-group, phase 3 trial. Lancet Infect Dis. 2023 October; 23(10):1119-1129. doi: 10.1016/S1473-3099 (23) 00299-2. Epub 2023 Jun. 8. Erratum in: Lancet Infect Dis. 2023 Sep. 1: PMID: 37302406.
Friedmann Angeli J P, et al. Inactivation of the ferroptosis regulator Gpx4 triggers acute renal failure in mice. Nat Cell Biol. 2014 December; 16(12):1180-91. doi: 10.1038/ncb3064. Epub 2014 Nov. 17. PMID: 25402683; PMCID: PMC4894846.
Zilka O, Shah R, Li B, Friedmann Angeli J P, Griesser M, Conrad M, Pratt D A. On the Mechanism of Cytoprotection by Ferrostatin-1 and Liproxstatin-1 and the Role of Lipid Peroxidation in Ferroptotic Cell Death. ACS Cent Sci. 2017 Mar. 22; 3(3):232-243. doi: 10.1021/acscentsci.7b00028. Epub 2017 Mar. 7. PMID: 28386601; PMCID: PMC5364454.
Bellanti F, Matteo M, Rollo T, De Rosario F, Greco P, Vendemiale G, Serviddio G. Sex hormones modulate circulating antioxidant enzymes: impact of estrogen therapy. Redox Biol. 2013 Jun. 19; 1(1):340-6. doi: 10.1016/j.redox.2013.05.003. PMID: 24024169; PMCID: PMC3757703.
Al-Hakeim H K, Al-Rubaye H T, Al-Hadrawi D S, Almulla A F, Maes M. Long-COVID post-viral chronic fatigue and affective symptoms are associated with oxidative damage, lowered antioxidant defenses and inflammation: a proof of concept and mechanism study. Mol Psychiatry. 2023 February; 28(2):564-578. doi: 10.1038/s41380-022-01836-9. Epub 2022 Oct. 24. PMID: 36280755; PMCID: PMC9589528.
Saha A K, Schmidt B R, Wilhelmy J, Nguyen V, Abugherir A, Do J K, Nemat-Gorgani M, Davis R W, Ramasubramanian A K. Red blood cell deformability is diminished in patients with Chronic Fatigue Syndrome. Clin Hemorheol Microcirc. 2019; 71(1):113-116. doi: 10.3233/CH-180469. PMID: 30594919; PMCID: PMC6398549.
Belikov A V, Schraven B, Simeoni L. T cells and reactive oxygen species. J Biomed Sci. 2015 Oct. 15; 22:85. doi: 10.1186/s12929-015-0194-3. PMID: 26471060; PMCID: PMC4608155.
Lopes, F., Coelho, F. M., Costa, V. V., Vieira, É. L., Sousa, L. P., Silva, T. A., Vieira, L. Q., Teixeira, M. M., and Pinho, V., Resolution of neutrophilic inflammation by H2O2 in antigen-induced arthritis, Arthritis Rheum., 2011, vol. 63, no. 9, pp. 2651-2660.
Medzhitov R, Schneider D S, Soares M P. Disease tolerance as a defense strategy. Science. 2012 Feb. 24; 335(6071):936-41. doi: 10.1126/science.1214935. PMID: 22363001; PMCID: PMC3564547.
Gupta S, et al. Sex differences in neutrophil biology modulate response to type I interferons and immunometabolism. Proc Natl Acad Sci USA. 2020 Jul. 14; 117(28):16481-16491. doi: 10.1073/pnas.2003603117. Epub 2020 Jun. 29. PMID: 32601182; PMCID: PMC7368314.
Soehnlein O, Steffens S, Hidalgo A, Weber C. Neutrophils as protagonists and targets in chronic inflammation. Nat Rev Immunol. 2017 April; 17(4):248-261. doi: 10.1038/nri.2017.10. Epub 2017 Mar. 13. PMID: 28287106.
Markman J L, Porritt R A, Wakita D, Lane M E, Martinon D, Noval Rivas M, Luu M, Posadas E M, Crother T R, Arditi M. Loss of testosterone impairs anti-tumor neutrophil function. Nat Commun. 2020 Mar. 31; 11(1):1613. doi: 10.1038/s41467-020-15397-4. PMID: 32235862; PMCID: PMC7109066.
Lu R J, Taylor S, Contrepois K, Kim M, Bravo J I, Ellenberger M, Sampathkumar N K, Benayoun B A. Multi-omic profiling of primary mouse neutrophils predicts a pattern of sex and age-related functional regulation. Nat Aging. 2021 August; 1(8):715-733. doi: 10.1038/s43587-021-00086-8. Epub 2021 Jul. 19. PMID: 34514433; PMCID: PMC8425468.
Lim E J, Ahn Y C, Jang E S, Lee S W, Lee S H, Son C G. Systematic review and meta-analysis of the prevalence of chronic fatigue syndrome/myalgic encephalomyelitis (CFS/ME). J Transl Med. 2020 Feb. 24; 18(1):100. doi: 10.1186/s12967-020-02269-0. PMID: 32093722; PMCID: PMC7038594.
Tomas C, Brown A, Strassheim V, Elson J L, Newton J, Manning P. Cellular bioenergetics is impaired in patients with chronic fatigue syndrome. PLoS One. 2017 Oct. 24; 12(10):e0186802. doi: 10.1371/journal.pone.0186802. Erratum in: PLoS One. 2018 Feb. 8; 13(2):e0192817. PMID: 29065167; PMCID: PMC5655451.
Choi B, et al. Persistence and Evolution of SARS-CoV-2 in an Immunocompromised Host. N Engl J Med. 2020 Dec. 3; 383(23):2291-2293. doi: 10.1056/NEJMc2031364. Epub 2020 Nov. 11. PMID: 33176080; PMCID: PMC7673303.
Taquet M, Dercon Q, Luciano S, Geddes J R, Husain M, Harrison P J. Incidence, co-occurrence, and evolution of long-COVID features: A 6-month retrospective cohort study of 273,618 survivors of COVID-19. PLoS Med. 2021 Sep. 28; 18(9):e1003773. doi: 10.1371/journal.pmed.1003773. PMID: 34582441; PMCID: PMC8478214.
Pretorius E, Venter C, Laubscher G J, Kotze M J, Oladejo S O, Watson L R, Rajaratnam K, Watson B W, Kell D B. Prevalence of symptoms, comorbidities, fibrin amyloid microclots and platelet pathology in individuals with Long COVID/Post-Acute Sequelae of COVID-19 (PASC). Cardiovasc Diabetol. 2022 Aug. 6; 21(1):148. doi: 10.1186/s12933-022-01579-5. PMID: 35933347; PMCID: PMC9356426.
Davis H E, Assaf G S, McCorkell L, Wei H, Low R J, Re'em Y, Redfield S, Austin J P, Akrami A. Characterizing long COVID in an international cohort: 7 months of symptoms and their impact. EClinicalMedicine. 2021 August; 38:101019. doi: 10.1016/j.eclinm.2021.101019. Epub 2021 Jul. 15. PMID: 34308300; PMCID: PMC8280690,
Zhang H, Wang L, Chu Y. Reactive oxygen species: The signal regulator of B cell. Free Radic Biol Med. 2019 October; 142:16-22. doi: 10.1016/j.freeradbiomed.2019.06.004. Epub 2019 Jun. 8. PMID: 31185253.
Komaroff A L, Lipkin W I. ME/CFS and Long COVID share similar symptoms and biological abnormalities: road map to the literature. Front Med (Lausanne). 2023 Jun. 2; 10:1187163. doi: 10.3389/fmed.2023.1187163. PMID: 37342500; PMCID: PMC10278546.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of determining the presence of a fatigue syndrome in an individual, the method comprising
obtaining a sample comprising circulating immune cells from the individual;
analyzing the population of immune cells for oxidative stress markers;
determining if the levels of the oxidative stress markers relative to a healthy control are indicative of the presence of a fatigue syndrome.

2. The method of claim 1, wherein the individual is treated in accordance with the classification.

3. The method of claim 1, wherein the individual is stratified for a clinical trial in accordance with the classification.

4. The method of claim 1, wherein a report of the classification is provided to the individual.

5. The method of claim 1, wherein the fatigue syndrome is selected from long Covid, myalgic encephalomyelitis and chronic fatigue syndrome (ME/CFS).

6. (canceled)

7. The method of claim 1, wherein the fatigue syndrome is an autoimmune disease, optionally systemic lupus erythematosus.

8. (canceled)

9. The method of claim 1, wherein the oxidative stress markers comprise markers for total reactive oxygen species levels; determination of the balance between oxidative stress and anti-oxidant pathways; and determination of total cellular oxidative damage.

10. The method of claim 9, wherein the markers for total reactive oxygen species levels comprise contacting the immune cells with a fluorogenic dye that reacts with oxygen species, and analyzing by flow cytometry for median and/or maximum fluorescence intensity.

11. The method of claim 9, wherein determination of the balance between oxidative stress and anti-oxidant pathways comprises one or more of determining glutathione (GSH) levels; determining the ratio of mitochondrial Ca²⁺ to superoxide dismutase 2 (SOD2); and determining expression of catalase.

12. The method of claim 1, wherein determination of total cellular oxidative comprises one or more of: detecting the expression of glutathione peroxidase 4 (GPX4); determining lipid peroxidation; determining changes in lipid droplet; determining changes in lysoPE phospholipid levels.

13. The method of claim 1, wherein a panel of oxidative stress markers comprises one or more of (a) determination of ROS by flow cytometry; (b) determination of the ratio of mitochondrial Ca++ to SOD2 mean fluorescence intensity by flow cytometry; (c) determination of the maximum glutathione (GSH) level by flow cytometry; (d) analyzing by flow cytometry the level of glutathione peroxidase 4 (GPX4); (e) determination of lipid peroxides; (f) determination of lipid droplets; and (g) lymphocyte proliferation in response to stimuli.

14. The method of claim 1, comprising performing a predictive classification algorithm relative to a training data set to determine if the levels of oxidative stress markers are indicative of the presence of a fatigue syndrome, wherein the clustering and predictive classification algorithm are analyzed by a computer processor comprising software configured for the purpose.

15. (canceled)

16. A method of screening a candidate agent for activity in treating a fatigue syndrome, the method comprising:
(a) contacting a cell population comprising lymphoid cells with a candidate agent;
(b) subjecting the cell population to induce ROS damage;
(c) labeling the cells with a detectable label for an oxidative stress marker;
(d) quantifying the levels of oxidative stress marker in the cell population;
wherein candidate agents for treatment of a fatigue syndrome shows reduced levels of oxidative stress.

17. The method of claim 16, wherein step (b) is performed prior to step (a).

18. The method of claim 16, wherein the cell population comprising lymphoid cells is an in vitro cultured cell line, or is a peripheral blood mononuclear cell population (PBMC).

19. (canceled)

20. The method of claim 18, wherein the PBMC are obtained from an individual diagnosed with a fatigue syndrome.

21. The method of claim 16, further comprising gating for lymphoid cells in step (d).

22. (canceled)

23. The method of claim 16, wherein the candidate agent is a small molecule drug, optionally an antioxidant.

24. (canceled)
